SEQUENCE LISTING (81 sequences)

SEQ ID NO: 1 - protein, 805 aa, Homo sapiens [sequence omitted]

SEQ ID NO: 2 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 4 - protein, 126 aa, Homo sapiens
DQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 5 - protein, 201 aa, Homo sapiens
PKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 31 aa, Homo sapiens
EPKSCDKTHTCPPCPAPELLGGPSVFLFPPK

SEQ ID NO: 7 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 22 aa, Artificial Sequence
  Synthetic construct
TSSGGGGENLYFQSSGGGSGGG

SEQ ID NO: 9 - protein, 19 aa, Artificial Sequence
  Synthetic construct
GSGGGGSGGGGSGGGGSPS

SEQ ID NO: 10 - protein, 22 aa, Artificial Sequence
  Synthetic construct
GSSGGGGSGGGGSGGGGSGGGG

SEQ ID NO: 11 - protein, 17 aa, Artificial Sequence
  Synthetic construct
GSSGGGGSGGGGSGGGG

SEQ ID NO: 12 - protein, 12 aa, Artificial Sequence
  Synthetic construct
GSSGGGGSGGGG

SEQ ID NO: 13 - protein, 11 aa, Artificial Sequence
  Synthetic construct
CSGGGGSGGGG

SEQ ID NO: 14 - protein, 15 aa, Artificial Sequence
  Synthetic construct
SGSETPGTSESATPE

SEQ ID NO: 15 - protein, 16 aa, Artificial Sequence
  Synthetic construct
SGSETPGTSESATPES

SEQ ID NO: 16 - protein, 15 aa, Artificial Sequence
  Synthetic construct
GGGGSGGGGSGGGGS

SEQ ID NO: 17 - protein, 50 aa, Artificial Sequence
  Synthetic construct
GGGGSGGGGSGGGGSGGGGSGGGGSGGGGSGGGGSGGGGSGGGGSGGGGS

SEQ ID NO: 18 - protein, 17 aa, Artificial Sequence
  Synthetic construct
AEAAAKEAAAKEAAAKA

SEQ ID NO: 19 - protein, 52 aa, Artificial Sequence
  Synthetic construct
AEAAAKEAAAKEAAAKEAAAKEAAAKEAAAKEAAAKEAAAKEAAAKEAAAKA

SEQ ID NO: 20 - protein, 5 aa, Artificial Sequence
  Synthetic construct
GGGGS

SEQ ID NO: 21 - protein, 16 aa, Artificial Sequence
  Synthetic construct
GLNDIFEAQKIEWHEG

SEQ ID NO: 22 - protein, 10 aa, Artificial Sequence
  Synthetic construct
EQKLISEEDL

SEQ ID NO: 23 - protein, 8 aa, Artificial Sequence
  Synthetic construct
HHHHHHHH

SEQ ID NO: 24 - protein, 19 aa, Artificial Sequence
  Synthetic construct
MRMQLLLLIALSLALVTNS

SEQ ID NO: 25 - protein, 7 aa, Artificial Sequence
  Synthetic construct
ENLYFQS

SEQ ID NO: 26 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - DNA, 8461 bp, Artificial Sequence
  Synthetic construct
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgcgaatgcagctgctgctgctgattgcgctgagcctggcgctggtgaccaacagcactagtcaatctaccatcgaagagcaggccaaaacattcctcgacttctttgatagccaggctgaagaccttttctaccaatcaagtctggctagctggaattacaatacaaacattacagaggagaacgtacaaaacatgaataacgcaggggacaagtggagcgcattccttaaggaacaaagtacccttgcgcaaatgtatccgctgcaagagattcaaaacctgacggttaagctgcaacttcaggccctccaacaaaatggatcctcagtcttgtcagaagacaaaagcaagcgactgaacaccatccttaacaccatgtcaaccatatattcaacaggtaaagtttgcaatccggataacccccaagaatgtttgcttcttgaacccggtctcaacgaaattatggccaacagtcttgattacaacgagcgattgtgggcatgggaaagttggaggagtgaggtaggcaaacagttgagacctctttatgaagagtacgttgtccttaaaaatgaaatggctcgcgcgaatcattatgaagactatggtgactactggaggggggattatgaggtgaacggggtggacggatacgattactctaggggccagctgatagaggatgtcgagcacacctttgaggagattaagccgttgtacgaacatttgcacgcctatgtcagggctaagctcatgaacgcttatccgagttatatctccccgataggatgcttgcctgctcacttgttgggcgatatgtggggacgcttttggaccaacttgtattcccttacggtaccgttcggccagaaaccaaatatcgacgtgacagacgcaatggtggatcaagcatgggatgcgcaacgaatcttcaaggaggcagaaaaatttttcgtttcagttggactcccaaacatgacgcagggtttctgggagaactcaatgttgacagatccaggtaatgtgcagaaagcggtttgcctccctactgcatgggatcttggtaaaggggacttccgcatactcatgtgtacgaaagtaactatggacgactttcttactgcgcaccacgagatggggcacatacaatacgatatggcgtacgcagctcaacctttccttctgcggaacggggcgaatgaaggatttcacgaggcagtgggtgagattatgtccctgtcagctgccactccgaaacatctgaaaagcatcggcctgttgagcccagacttccaagaagataatgagaccgaaataaacttccttctgaagcaagcactgactattgtaggtaccttgccctttacatacatgctggagaagtggaggtggatggtatttaagggggagataccgaaagatcaatggatgaaaaagtggtgggaaatgaaaagggagatcgttggcgtagttgaaccagtaccgcatgatgagacgtactgcgatccggctagtctgttccatgtctctaatgattactctttcatccgctactacacccgcacgctgtatcaattccagttccaagaagctctctgtcaggctgccaagcacgaaggaccgctgcacaaatgcgacattagcaattctacagaggcgggtcagaagttgttcaatatgcttagactggggaagagcgaaccgtggacgctcgctttggagaacgttgttggagctaagaatatgaacgtcaggcccttgctgaattactttgaacctctgtttacgtggttgaaagaccaaaataaaaactcctttgttgggtggagtactgactggtccccctatgcggaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccactagttctggtggtggtggtgagaatctgtactttcagagctcgggcggaggatcgggtggaggcgagcccaaatcttgtgacaaaactcacacatgccccccctgcccagcgccagaattgctgggcggacccagcgtgttcctgttcccccccaaacctaaagacaccctgatgatcagccgaacccctgaggtgacctgcgtggtggtggacgtgagccacgaggaccccgaggtgaagttcaactggtatgtggacggcgtggaggtccacaatgccaaaacgaagcccagggaggagcagtacaacagcacctacagggtagtgagcgtcttgaccgtgctgcaccaggactggctgaacggcaaggaatacaaatgcaaggtcagcaataaggctctgccggctcctatcgagaagacaatcagcaaggcaaagggccagccacgcgaaccgcaggtgtatactctgccccccagccgggacgagctgaccaagaaccaggtgtccctgacctgtctggtgaaaggcttctaccccagcgacatcgctgtggagtgggagagtaacgggcagcccgagaacaactacaagaccacgcctcctgtgctggacagcgacggcagcttcttcctgtatagcaagctcaccgtggacaagagcaggtggcaacagggcaacgtgttcagctgctctgtgatgcacgaggccctgcacaaccattacacccagaagagtctcagtctgagcccgggaaagggtggaggcggatccggcctgaacgacatcttcgaggctcagaaaatcgaatggcacgaaggctaatcccagtgtcgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgattagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgccaccatgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaataatctagagggcccgcggttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcgggtgcaagtgcaggtgccagaacatttctctatcgaa

SEQ ID NO: 28 - protein, 1256 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 29 - protein, 193 aa, Severe acute respiratory syndrome coronavirus
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 30 - DNA, 5566 bp, Artificial Sequence
  Synthetic construct
atgtatagaatgcaactcctgtcctgcatcgccctctccctggcactggtgactaacagtaacattactaacctttgccccttcggcgaggttttcaatgccaccaaatttccatctgtctatgcatgggaacggaagaaaatctccaactgcgtcgctgattattcagtgctgtacaactcaaccttcttttcaacgtttaagtgttatggcgttagtgccacgaagctcaacgacttgtgcttttccaatgtctacgccgattcttttgtggtcaagggagatgacgtgcgccaaatagcacccggacaaacaggagtcattgctgattataactataaactccctgacgactttatgggttgtgtactcgcatggaacactagaaacattgatgcgacatcaaccggtaactataattataaatatcgatatctgcgccacgggaaacttcggcctttcgaaagagacataagcaatgttccttttagcccagacggcaaaccctgtacaccaccagccctcaattgctattggcccctgaacgactatgggttctataccaccaccggcataggctaccaaccctacagggtggtggtcctcagttttgagttgcttaatgctccggctacagtgggcggcggcggcagcggggggggtggtagtcatcatcatcatcaccaccaccatggattgaatgatatctttgaggcccagaagatcgaatggcatgaataggctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcaaaggaggtaccgcgatatctacctcgag

SEQ ID NO: 31 - protein, 136 aa, Human coronavirus NL63
QHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGI

SEQ ID NO: 32 - DNA, 5443 bp, Artificial Sequence
  Synthetic construct
atggggatcttaccatctcctgggatgcccgccctgctgtctctggtgagtcttttgagcgtcctccttatgggttgtgtagcggagaccggcacccagcatacagacattaacttcacagccacagcaagtttcggaggctcttgttatgtgtgtaaacctcaccaggtgaatatctctttaaacggtaatacctctgtctgtgtacgaaccagccacttctctattcggtacatctataatcgtgtgaagtcaggatctccaggcgattcttcctggcacatctacctgaaatctggaacctgccccttctcttttagcaaactgaacaacttccaaaagtttaaaacaatctgctttagcacggtggaggtgcctgggtcatgtaacttccctttggaggctacttggcactacaccagttacaccattgtcggcgctctgtacgtaacatggtcagaagggaattcaatcaccggcgtcccctatccggtgagcgggatcggtggaggtggcagtggcggaggaagcgggggcggtggcagtcatcatcaccaccatcatcaccatggcctgaatgatatctttgaggctcagaagattgaatggcacgagtgagctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcaaaggaggtaccgcgatatctacctcgag

SEQ ID NO: 33 - protein, 206 aa, Severe acute respiratory syndrome coronavirus 2
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 34 - DNA, 22 bp, Artificial Sequence
  Synthetic construct
tggatcagcaagcaggagtatg

SEQ ID NO: 35 - DNA, 18 bp, Artificial Sequence
  Synthetic construct
gcatttgcggtggacgat

SEQ ID NO: 36 - DNA, 21 bp, Artificial Sequence
  Synthetic construct
caatggtttaacaggcacagg

SEQ ID NO: 37 - DNA, 21 bp, Artificial Sequence
  Synthetic construct
ctcaagtgtctgtggatcacg

SEQ ID NO: 38 - protein, 740 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 39 - protein, 729 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQP

SEQ ID NO: 40 - protein, 227 aa, Artificial Sequence
  Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 88 aa, Artificial Sequence
  Synthetic construct
QSTIEEQARTFLDFFNIQAEDLSYQSSLASWNHNTNITEENVQNMNNAGDKWSAFLKKQSTLAQMYPLQEIQNLTVKLQLQALQQNGS

SEQ ID NO: 42 - protein, 88 aa, Artificial Sequence
  Synthetic construct
QSTIEEQAKTFLDFFNIQAEDLFYQSSLASWNYNTDITEENVQNMNNAGDKWSAFLKEQSTLAQMYPLQEIRNLAVKLQLQALQQNGS

SEQ ID NO: 43 - protein, 88 aa, Artificial Sequence
  Synthetic construct
QSTIEEQAKTFLDFFNIQAEDLFYQSSLASWNYNTNTTEKNVQNVNNAGDKWSAFLKEQGTLAQMYPLQEIQQPTVKLQLQALQQNGS

SEQ ID NO: 44 - protein, 88 aa, Artificial Sequence
  Synthetic construct
QSTIEEQAKTFLDFFNIQAEDLFYQSSLASWNYNTNITEENVQNMNNAGDKWSAFLKEQSTLAQMYPLQEIQDPTVKLQLQALQQNGS

SEQ ID NO: 45 - protein, 88 aa, Artificial Sequence
  Synthetic construct
QSTIEEQAKTFLDFFNIQAEDLFYQSSLASWNYNTNITEENVQNMNNAGDKWSAFLKEQSTLAQMYPLQEIQNPTVKLQLQALQQNGS

SEQ ID NO: 46 - protein, 88 aa, Artificial Sequence
  Synthetic construct
QSTIEEQAKTFLDFFNIQAEDLFYQSSLASWNYNTNITEESVQNMNNAGDKWSAFLKEQSTLAQMYPLQEIQNPTVKLQLQALQQNGS

SEQ ID NO: 47 - protein, 88 aa, Artificial Sequence
  Synthetic construct
QSTIEEQAKTFLDFFNIQAEDLFYQSSLASWNYNTNITEENVQNMNNAGDKWSALLKEQSTLAQMYPLQEIQNLTVKLQLQALQQNGS

SEQ ID NO: 48 - protein, 88 aa, Artificial Sequence
  Synthetic construct
QSTIEEQAKTFLDFFNAQAEDLFYQSSLASWNYNTNITEENVQNMDNAGDKWSAFLKEQSTLAQMYPLQEIQNLTVKLQLQALQQNGS

SEQ ID NO: 49 - protein, 88 aa, Artificial Sequence
  Synthetic construct
QSTIEEQAKTFLDFFNIRAEDLFYQSSLASWNYNTNITEENVQNMNNAGDRWSAFLKEQSTLAQMYPLQEIQNLTVKLQLQALQQNGS

SEQ ID NO: 50 - protein, 88 aa, Artificial Sequence
  Synthetic construct
QSTVEEQAKTFLDFFNIRAEDLFYQSSLASWDYNTNITEENVQNMNNAGDKWSAFLKEQSTLAQMYPLQEIQNLTVKLQLQALQQNGS

SEQ ID NO: 51 - protein, 88 aa, Artificial Sequence
  Synthetic construct
QSTIEEQAKTFLDKFNVEAEDLFYQSSLASWNYNTNVTEENVRNMNNAGDKWSAFLKEQSTLAQMYPLQEIQNLTVKLQLQALQQNGS

SEQ ID NO: 52 - protein, 88 aa, Artificial Sequence
  Synthetic construct
QPTIEEQAKTFLDKFSVEAEDLLYQSSLASWDYNTNITEENVQNMNNAGDKWSAFLKEQSTLAQMYPLQEIQNLTVKLQLQAPQQNGS

SEQ ID NO: 53 - protein, 88 aa, Artificial Sequence
  Synthetic construct
QSTIEEQVKAFLDKFNAEAEDLFYQSSLASWSYNTNITEANVQNMNNAGDKWSAFLKEQSTLAQMYPLQEIQNLTVKLQLQALQQNGS

SEQ ID NO: 54 - protein, 88 aa, Artificial Sequence
  Synthetic construct
QSTIEEQVKAFLDKFNAEAEDLLYQSSLASWNCNTNITEENVQNMNNAGDKWSAFLKEQSTLAQMYPLQEIQNLTVKLQLQALQQNGS

SEQ ID NO: 55 - protein, 88 aa, Artificial Sequence
  Synthetic construct
QSTIEEQAKTFLDKFNHEAEDLFYQSSLASWNYNTNITEENVQNMNNAGDKWSAFLREQSTLAQMYPLQEIQQLTVKLQLQALRQNGS

SEQ ID NO: 56 - protein, 88 aa, Artificial Sequence
  Synthetic construct
QSTIEEQAKTFLDKFNAEAEDLSYRSSLASWDYNTNISEENVQNMNNAGDKWSAFLKEQSTLAQMYPLQEIQQLTVKLQLQALRQNGS

SEQ ID NO: 57 - protein, 88 aa, Artificial Sequence
  Synthetic construct
QSTIEEQAKTFLDKFNVEAEDLFYQSSLASWNYNTNITEENVQNMNNAGDKWSAFLKEQSTLAQMYPLQEIQQLTVELQLQALQQNGS

SEQ ID NO: 58 - protein, 88 aa, Artificial Sequence
  Synthetic construct
QSTIEEQAKTFLDFFNIQAEDLLYQSSLASWNYNTNITEENVQNMNNAGDKWSAFLKEQSTLAQMYPLQEIPNLTVKLRLQALQQNGS

SEQ ID NO: 59 - protein, 88 aa, Artificial Sequence
  Synthetic construct
QSTIEEQAKTFLDFFNIQAEDLFYRSSLASWNYNTNITEENVQNMNNAGDKRSAFLKEQSTLAQMYPLQEIQQLTVKLQLQALRRNGS

SEQ ID NO: 60 - protein, 88 aa, Artificial Sequence
  Synthetic construct
QSTIEEQAKAFLDKFNAEAEDLFYQSSLASWSYNTNITEENAQNMSNAGDRWSAFLKGQSTLAQMYPLQEIQQLTVKLQLQALQRNGS

SEQ ID NO: 61 - protein, 88 aa, Artificial Sequence
  Synthetic construct
QPTIEEQARAFLDKFNAEAEDLFYQSGLASWNYNTNITEENVQNTNNAGDKWSAFLKEQSTLAQMYPLQEIQNLTVKLQLQALQQNGS

SEQ ID NO: 62 - protein, 88 aa, Artificial Sequence
  Synthetic construct
PSTIEEQAKTFLDFFDIQAEDLFYQSSLVSWNYNTNITEENVQNMNNAGDTWSAFLKEQNTPAQMYPLQEIQNLTVKRQLQALQQNGS

SEQ ID NO: 63 - protein, 88 aa, Artificial Sequence
  Synthetic construct
QSTIEEQAKTFLDFFDTQAEDLFYQSSLASWNYNTDITEENVQNMNNAGDKRSAFLKKQSTLAQMYPLQEIQNLTVKLQLQALQQNGS

SEQ ID NO: 64 - protein, 88 aa, Artificial Sequence
  Synthetic construct
RSTIEEQAKTFLDFFDSQAEDLFYQSSLASWNYNTNITEENVQNMNNAGDKRSAFLKERSTLAQMYPLQEIQNLTVKLQLQALQQNGS

SEQ ID NO: 65 - protein, 88 aa, Artificial Sequence
  Synthetic construct
QSTIEEQAKTFLDFFNIQAEDLFYQSSLASWNYNTNITEENVQNINDAGGKWSAFLKEQSTLAQMYPLQEIQDLTVKLQLQALQQNGS

SEQ ID NO: 66 - protein, 88 aa, Artificial Sequence
  Synthetic construct
QSTIEEQAKTFLDFFSIRAEDLFYQSSLASWNYNTNITEENVRNVNNAGDKWSAFLKEQSTLAQMYPLQEIQNLTVKLQLQALQQNGS

SEQ ID NO: 67 - protein, 88 aa, Artificial Sequence
  Synthetic construct
QSTIEEQAKTFLDFFNIQAEDLLYQSSLASWNYNTNITEENVQNMNNAGDKWSAFLKEQSTLAQMYPLQEIQNLTVKLQLQAPQQNGS

SEQ ID NO: 68 - protein, 88 aa, Artificial Sequence
  Synthetic construct
QSTIEEQAKTFLDFFNVQAEDLSYQSSMASWNYNTNITEENVQNMNNAGDKWSAFLKEQSTPAQMYPLQEIQNLTVKLQLQALQQNGS

SEQ ID NO: 69 - protein, 88 aa, Artificial Sequence
  Synthetic construct
QSTIEEQAKTFLDFFNIQAEDLFYQSSLASWNYNTNITEENVQNMNNAGDKWSAFLKEQSTPAQMYPLQEIQNLTVKLQLQALQQNGS

SEQ ID NO: 70 - protein, 88 aa, Artificial Sequence
  Synthetic construct
QSAIEEQAKTFLDFFNIQAEDLFYQSSLASWNYNTNITEENVQNMNNAGDKWSAFLKEQSTFAQMYPLQEIQNLTVKLQLQALQQNGS

SEQ ID NO: 71 - protein, 88 aa, Artificial Sequence
  Synthetic construct
QSTIEEQAKTFLDFFDTQAEDLFYQSSLASWNYNTNITEENVQNMNNAGDKWSAFLKEQSTLAQMYPLQEIQNLTVKLQLQALQQNGS

SEQ ID NO: 72 - protein, 88 aa, Artificial Sequence
  Synthetic construct
QSTIEEQAKTFLDFFSVQAEDLFYQSSLASWNYNTNITEENVQNMNNVGDKWSAFLKEQSTLAQMYPLQEIQNLTVKLQLQALQQNGS

SEQ ID NO: 73 - protein, 88 aa, Artificial Sequence
  Synthetic construct
QSTIEEQAKTFLDFFSIQAEDLFYQSSLASWNYNTNITEENVQNMNNAGDKWSAFLKEQSTLAQMYPLQEIQNLTVKLQLQALQQNGS

SEQ ID NO: 74 - protein, 88 aa, Artificial Sequence
  Synthetic construct
QSTIEEQAKTFLDFFNIQAEDLFYQSSLASWNYNTNITEENVQNMNNAGDKWSAFLKERSTLAQMYPLQEIQNLTVKLQLQALQQNGS

SEQ ID NO: 75 - protein, 88 aa, Artificial Sequence
  Synthetic construct
QSTIEEQAKTFLDFFNIQAEDLFYQSSLASWNYNTNITEENVQNMNNAGDKWSAFLKEQSTLAQMYPLQEIQNLTVKLQLQALQQNGS

SEQ ID NO: 76 - protein, 80 aa, Artificial Sequence
  Synthetic construct
QSTIEEQAKTFLDLSNLQAEDLFYQSGLASWNYNTNITGENVQNTDNAGDKWSAFLKEQSTLAQMYPLQEIQNLTVKLQL

SEQ ID NO: 77 - protein, 994 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSSGGGGSGGGGSGGGGSGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 989 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSSGGGGSGGGGSGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 984 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGSSGGGGSGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 983 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSCSGGGGSGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 958 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0257726 A1
(43) Pub. Date: Aug. 17, 2023

(54) ACE2 COMPOSITIONS AND METHODS

(71) Applicants: Chan Zuckerberg Biohub, Inc., San Francisco, CA (US); The Regents of the University of California, Oakland, CA (US)

(72) Inventors: Anum Glasgow, Oakland, CA (US); Jeff Glasgow, Oakland, CA (US); James A. Wells, Oakland, CA (US); Xin Zhou, Oakland, CA (US); Tanja Kortemme, Oakland, CA (US); Irene Lui, Oakland, CA (US)

(21) Appl. No.: 17/997,877

(22) PCT Filed: May 11, 2021
(86) PCT No.: PCT/US21/31828

§ 371 Date: Nov. 3, 2022

Related U.S. Application Data
(60) Provisional application No. 63/022,789, filed on May 11, 2020.
(60) Provisional application No. 63/056,509, filed on Jul. 24, 2020.
(60) Provisional application No. 63/058,379, filed on Jul. 29, 2020.
(60) Provisional application No. 63/067,273, filed on Aug. 18, 2020.

(51) Int. Cl. C12N 9/48; C12N 15/86; A61P 31/14

(52) U.S. Cl. CPC C12N 9/485; C12Y 304/17023; C12N 15/86; A61P 31/14; C07K 2319/30; C12N 2770/20022

(57) ABSTRACT

This disclosure describes recombinant angiotensin-converting enzyme II (ACE2) polypeptides, fusion proteins, and compositions thereof having improved binding affinity for the SARS-CoV-2 spike protein receptor binding domain relative to wild-type ACE2. Also provided are methods of using the recombinant ACE2 polypeptides, fusion proteins, and compositions thereof for treating subjects infected with a SARS-CoV-2 virus (i.e., subjects with COVID-19), subjects having symptoms suggestive of a SARS-CoV-2 infection, and subjects exposed to or at risk of exposure to SARS-CoV-2 virus. Other virus infections may also be treated.

### CROSS-REFERENCES TO RELATED APPLICATIONS

This application claims priority to U.S. Provisional Application No. 63/022,789, filed on May 11, 2020; U.S. Provisional Application No. 63/056,509, filed on Jul. 24, 2020; U.S. Provisional Application No. 63/058,379, filed on Jul. 29, 2020; and U.S. Provisional Application No. 63/067,273, filed on Aug. 18, 2020. The entire disclosure of each of the aforementioned provisional applications is herein incorporated by reference for all purposes.

### STATEMENT AS TO RIGHTS TO INVENTIONS MADE UNDER FEDERALLY SPONSORED RESEARCH AND DEVELOPMENT

This invention was made with government support under grant no. K99 GM135529 awarded by The National Institutes of Health. The government has certain rights in the invention.

### SEQUENCE LISTING

The official copy of the sequence listing is submitted electronically via EFS-Web as an ASCII formatted sequence listing with a file named 1244650 seqlist.txt, created on May 10, 2021, and having a size of 166 KB, and is filed concurrently with the specification. The sequence listing contained in this ASCII formatted document is part of the specification and is herein incorporated by reference in its entirety.

### BACKGROUND

Coronaviruses (CoV) are a large family of viruses that cause illness ranging from the common cold to more severe diseases such as Middle East Respiratory Syndrome (MERS-CoV) and Severe Acute Respiratory Syndrome (SARS-CoV). Coronaviruses are zoonotic, meaning they can be transmitted between animals and humans.

Severe Acute Respiratory Syndrome Coronavirus 2 (SARS-CoV-2) is the strain of coronavirus that causes coronavirus disease 2019 (COVID-19), a respiratory illness. It was initially referred to by its provisional name, 2019 novel coronavirus (2019-nCoV). SARS-CoV-2 has spread throughout the world and, as of now, has resulted in over 153 million cases of COVID-19 and over 3.2 million deaths. SARS-CoV-2 primarily spreads between people through close contact and via respiratory droplets produced from coughs or sneezes.

Epidemiological studies estimate each infection results in 1.4 to 3.9 new ones when no members of the community are immune and no preventive measures taken. Several variants of SARS-CoV-2 have been identified, including B.1.1.7, also known as the “UK variant,” initially detected in the United Kingdom, and B.1.351, also known as the “South Africa variant,” initially detected in South Africa in December 2020.

SARS-CoV-2 is an enveloped positive-sense RNA virus. This virus is characterized by club-like spikes on the surface. The virus can enter eukaryotic cells via endosomes or plasma membrane fusion. In both routes, spikes on the virion surface bind to the membrane-bound receptor ACE2 and mediate attachment to the membrane of and entry into a host cell.

To date, only one active pharmaceutical agent, remdesivir (Gilead Sciences), has demonstrated any clinical effect in treating COVID-19 patients, and it promotes only a modest reduction in time to recovery in severely ill patients without showing any statistically significant reduction in mortality. There is an urgent and ongoing need for agents to treat COVID-19 and Coronavirus infections in general.

### BRIEF SUMMARY

Provided herein are recombinant angiotensin-converting enzyme II (ACE2) polypeptides and compositions thereof having improved binding affinity for the SARS-CoV-2 spike receptor binding domain compared to wild-type ACE2.

In one aspect, provided is a recombinant ACE2 polypeptide comprising a soluble ACE2 receptor ectodomain polypeptide comprising an amino acid sequence having at least 90% sequence identity to the amino acid sequence as set forth in SEQ ID NO: 2 or 3 and comprising at least one amino acid residue substitutions selected from the group consisting of Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, and Q101R, wherein the residues are numbered with reference to SEQ ID NO:1.

In some embodiments, the soluble ACE2 receptor ectodomain polypeptide comprises at least two amino acid residue substitutions selected from the group consisting of Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, and Q101R, wherein the residues are numbered with reference to SEQ ID NO:1.

In some embodiments, the soluble ACE2 receptor ectodomain polypeptide comprises amino acid residue substitutions:

i. K31F, N33D, H34S, and E35Q;

ii. K31F, N33D, H34A, E35Q, N49D, N51S, N53S, E57G, and N64D;

iii. T27A, K31F, N33D, H34S, E35Q, N61D, K68R, and L79P;

iv. S19P, N33S, H34V, F40L, N49D, and L100P;

v. K31F, N33D, H34S, E35Q, W69R, and Q76R;

vi. Q18R, K31F, N33D, H34S, E35Q, W69R, and Q76R;

vii. Q18R, K31F, N33D, H34S, E35Q, W69V, and Q76R;

viii. Q18R, K31F, N33D, H34S, E35Q, W69K, and Q76R;

ix. Q18R, K31F, N33D, H34S, E35Q, W69I, and Q76R;

x. T27A, H34A, N49S, V59A, N63S, K68R, E75G, N90Q, and Q103R;

xi. K31F, N33D, H34T, N53D, W69R, and E75K;

xii. S19P, K26R, T27A, H34A, S44G, and M62T;

xiii. K31F, H34I, E35Q, and N90Q;

xiv. A25V, T27A, H34A, and F40D;

xv. K31Y, W69V, L79T, and L91P;

xvi. T27Y, H34A, and N90Q;

xvii. S19P, Q42L, L79T, and N90Q;

xviii. K31F, H34I, E35Q; or

xix. H34V and N90Q,

wherein the residues are numbered with reference to SEQ ID NO:1.

In some embodiments, the soluble ACE2 receptor ectodomain polypeptide comprises amino acid residue substitutions H374N and H378N.

In some embodiments, the soluble ACE2 receptor ectodomain polypeptide comprises amino acid residue substitution H345L.

In another aspect, provided is a fusion protein comprising a recombinant ACE2 polypeptide as described above fused to a dimerization domain. In some embodiments, the recombinant ACE2 polypeptide is fused to the dimerization domain via a peptide linker. In some embodiments, the dimerization domain comprises an Fc domain.

In another aspect, provided is a recombinant nucleic acid encoding a recombinant ACE2 polypeptide protein or a fusion protein as described above.

In another aspect, provided is a DNA construct having a promoter operably linked to the recombinant nucleic acid. In some embodiments, the promoter is a heterologous promoter.

In another aspect, provided is a vector comprising the DNA construct described above.

In another aspect, provided is a host cell comprising the recombinant nucleic acid described above.

In another aspect, provided is a host cell comprising the DNA construct described above.

In another aspect, provided is a host cell comprising the vector described above.

In some embodiments, any of the host cells described above can be a eukaryotic cell.

In another aspect, provided is a composition comprising a dimer of the recombinant ACE2 polypeptide or the fusion protein described above.

In another aspect, provided is a method of producing a recombinant ACE2 polypeptide comprising culturing any host cell as described above under conditions sufficient for the production of the recombinant ACE2 polypeptide by the host cell.

In another aspect, provided is a pharmaceutical preparation comprising: (a) the recombinant ACE2 polypeptide or the fusion protein as described above; and (b) a pharmaceutically acceptable carrier.

In another aspect, provided is a method for treating a subject infected with a SARS-CoV-2 virus or having symptoms suggestive of a SARS-CoV-2 infection, the method comprising administering to a subject a therapeutically effective amount of the pharmaceutical preparation as described above. In some embodiments, the subject has a confirmed SARS-CoV-2 infection.

In another aspect, provided is a method for treating a subject exposed to a SARS-CoV-2 virus or at risk of exposure to SARS-CoV-2 virus, the method comprising administering to a subject a therapeutically effective amount of the pharmaceutical preparation as described above.

In some embodiments of the provided methods, the subject is human.

In some embodiments of the provided methods, the pharmaceutical preparation is administered intravenously.

In some embodiments of the provided methods, the pharmaceutical preparation is administered at least once per day.

In some embodiments, the recombinant ACE2 polypeptide, the fusion protein, and/or the composition as described above has increased binding affinity for SARS-CoV-2 spike RBD relative to wild-type human ACE2 protein ectodomain.

In some embodiments, the recombinant ACE2 polypeptide, the fusion protein, and/or the composition as described above has greater than 180-fold higher affinity for SARS-CoV-2 spike RBD as compared to wild-type human ACE2 protein ectodomain.

In some embodiments, the recombinant ACE2 polypeptide, the fusion protein, and/or the composition as described above has increased efficacy in neutralizing SARS-CoV-2 virus compared to wild-type human ACE2 protein ectodomain.

In some embodiments, the recombinant ACE2 polypeptide, the fusion protein, and/or the composition as described above has greater than 25-fold higher neutralization efficacy for SARS-CoV-2 virus compared to wild-type human ACE2 protein ectodomain.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows plasmid constructs for expression of ACE2(614) (top row), ACE2(740) (second row), and spike, in the monomeric RBD (third row) and full-length (FL) (bottom row) forms, according to certain aspects of this disclosure. Abbreviations: IL2 SS, IL2 signal sequence (cleaved); TEV-linker, TEV protease cleavage sequence and glycine-serine linker; hIgG1-Fc, human IgG1 Fc domain; avi tag, target sequence for intracellular biotinylation by BirA; 8×His tag, 8× polyhistidine tag.

FIG. 2 shows that increased stability, affinity, and avidity effects result more potent viral neutralization, according to certain aspects of this disclosure. The top panel shows a table listing all ACE2 variants with scaffolds and mutations tested in pseudotyped and authentic SARS-CoV-2 viral neutralization assays with their origin. The bottom panel shows that Fc fusion, inclusion of the collectrin domain, and affinity-enhancing mutations improve neutralization of ACE2 constructs against pseudotyped SARS-CoV-2 virus, except for misfolded variant 353, according to certain aspects of this disclosure. Error bars represent standard deviations over all technical replicates from 2-4 biological replicates. Biological replicates were separate experiments using different preparations of ACE2 variant and pseudovirus, each with 2 or 4 technical replicates. Statistical significance with P<0.01 was determined using a homoscedastic two-tailed t test.

FIG. 3 shows that Fc fusion, addition of computationally predicted and DMS-guided mutations, inclusion of the collectrin domain, and mutations from affinity maturation in yeast enhance potency of neutralization of authentic SARS-CoV-2 virus, according to certain aspects of this disclosure. ACE2 constructs on the X-axis are described in FIG. 2 (top panel). The top panel shows that that Fc fusion (variant 13) and addition of computationally-predicted mutations (variant 118) enhance neutralization by >50,000-fold over a control anti-GFP IgG antibody, according to certain aspects of this disclosure. Error bars represent standard deviation of the mean for biological duplicates. The bottom panel shows that inclusion of the collectrin domain, computationally predicted mutations (variant 293), DMS-guided mutations (variant 310), and mutations from affinity maturation in yeast (variant 313) enhance neutralization potency over a control anti-GFP IgG antibody sample to IC50 values of 73 to 136 ng/mL, according to certain aspects of this disclosure. Error bars represent standard error of the mean for biological duplicates.

FIG. 4 shows that the ACE2 variants are not strongly cytotoxic in uninfected VeroE6 cells after 24 hours of treatment, according to certain aspects of this disclosure. ATP release was measured by luminescence signal using the CellTiter-Glo® assay (Promega) in ACE2 variants-treated cells relative to anti-GFP IgG-treated cells. Error bars represent standard error of the mean for biological duplicates.

FIG. 5 shows alignment of sequences of receptor binding domains from SARS-CoV-2 (SEQ ID NO:33; labelled “CoV-2”), SARS-CoV-1 (SEQ ID NO:29; labelled “CoV-1”), and HCoV-NL63 (SEQ ID NO:31; labelled “NL63”) spike proteins, according to certain aspects of this disclosure. Numbers flanking each row indicate residue positions for each RBD sequence in the respective full-length, wild-type spike protein.

FIG. 6 shows that wild-type ACE2 effectively hydrolyzes angiotensin II (Ang-II) using mass spectrometry, according to certain aspects of this disclosure. Angiotensin II has a mass of 1046.54 Da, and cleavage by ACE2 produces Angiotensin(1-7) having a mass of 899.49. Both peptides have sodium adducts adding 22 Da. CVD208 (wild-type ACE2(740)-Fc) (1 μg/mL) hydrolyzed >90% of Ang-II (200 μM) over 60 min at 37° C., as indicated by the relative intensities of the large peak at 1046.54 m/z (“Ang-II”) and the much smaller peak at 899.49 m/z (“Ang(1-7)”).

FIG. 7 shows that an ACE2 variant with an inactivating mutation does not effectively hydrolyze Ang-II using mass spectrometry, according to certain aspects of this disclosure. CVD313 (ACE2(740)-Fc (K31F/N33D/H34S/E35Q/H345L) shows virtually no detectable hydrolysis of Ang II under the same conditions used in FIG. 6, as indicated by the very small peak at 899.47 (“Ang(1-7)”) relative to the much larger peak at 1046.54 (“Ang-II”). Inset shows zoomed-in region containing Ang(1-7), with very little formation from CVD313.

FIG. 8 shows that an ACE2 variant with an inactivating mutation does not effectively hydrolyze Ang-II using mass spectrometry, according to certain aspects of this disclosure. CVD118 (ACE2(614)-Fc, H34V/N90Q/H374N/H378N) shows virtually no detectable hydrolysis of Ang II under the same conditions used in FIGS. 6 and 7, as indicated by the lack of a peak at 899.47 (“Ang(1-7)”) and a large peak at 1046.54 (“Ang-II”). Inset shows zoomed-in region containing Ang(1-7).

FIG. 9 shows that enzymatically active ACE2 variants bind spike RBD and full-length spike, according to certain aspects of this disclosure. Variants are detailed in Table 4 and Table 9 herein. The data shown are a snapshot of the dissociation curve of the variant being tested. A value of 1 for fraction bound (when the data are normalized to the no inhibitor condition) means that the protein has not appreciably dissociated over the course of the experiment. A value of 0.5 would indicate that one complex half life has passed. A value of 0 would indicate that the protein has completely dissociated. The fraction of each ACE2 variant bound to full-length spike (S6P; left bar in graph for each variant) and RBD (right bar in graph for each variant) is shown at 1.5 hr (top panel) and 6 hr (bottom panel).

FIG. 10 shows that a receptor trap construct with an enzymatically active ACE2 domain binds various mutated spike proteins, including those from the UK and South Africa SARS-CoV-2 variants, according to certain aspects of this disclosure. The data shown are a snapshot of the dissociation curve of the variant being tested. A value of 1 for fraction bound (when the data are normalized to the no inhibitor condition) means that the protein has not appreciably dissociated over the course of the experiment. A value of 0.5 would indicate that one complex half life has passed. A value of 0 would indicate that the protein has completely dissociated. The fraction of the ACE2 receptor trap bound to each full-length spike (S6P) is shown at 2 hr.

### DETAILED DESCRIPTION

### I. Introduction

Provided herein are recombinant angiotensin-converting enzyme II (ACE2) polypeptides and compositions thereof having improved binding affinity for the SARS-CoV-2 spike receptor binding domain compared to wild-type ACE2. The recombinant ACE2 polypeptides provided are ACE2 receptor traps: engineered, soluble variants of the ACE2 extracellular domain that mimic the viral interaction with the human receptor in the serum to block the virus from binding cellular ACE2, gaining entry into cells, and propagating. In some instances, the recombinant ACE2 polypeptides are fusion proteins comprising an ACE2 ectodomain variant sequence and a dimerization domain. Compositions and pharmaceutical preparations of recombinant ACE2 polypeptide dimers are also provided as are DNA constructs, vectors, host cells, and associated methods. Also provided are methods of using the recombinant ACE2 polypeptides for treating subjects infected with a SARS-CoV-2 virus or other coronavirus, subjects having symptoms suggestive of a SARS-CoV-2 infection or other coronavirus infection, subjects exposed to a SARS-CoV-2 virus or other coronavirus, and/or subjects at risk of exposure to SARS-CoV-2 virus or other coronavirus.

Full-length human ACE2 is 805 amino acids in length (SEQ ID NO:1), of which amino acids 1-17 is a signal peptide that is cleaved from the mature protein. See NCBI Reference Sequence NP_001358344.1; see also UniProtKB Reference Q9BYF1. The ACE2 ectodomain (SEQ ID NO: 2) is composed of a N-terminal peptidase domain (aa 18-614) (SEQ ID NO:3) and a C-terminal dimerization domain, also referred to as a “collectrin” domain (aa 615-740) (SEQ ID NO:4). Recent studies have revealed the structural basis of the high-affinity ACE2-spike interaction through the spike receptor binding domain (RBD) (Lan, J., et al., Nature, 581:215-220 (2020) and Yan, R., et al., Science, 367(6485):1444-1448 (2020)). The ACE2-RBD co-structure shows a large, flat binding interface primarily comprising the N-terminal helices of ACE2 (residues 18-90), with secondary interaction sites spanning residues 324-361. The inventors determined that binding affinity of the ACE2-spike interaction is further improved through intermolecular avidity effects, as demonstrated by the efficacy of engineered dimeric ACE2-Fc fusion proteins in neutralizing SARS-CoV-2. See U.S. provisional patent application 63/022,789 and Lui, I., et al. bioRxiv, doi.org/10.1101/2020.05.21.109157, published May 21, 2020, both of which are incorporated herein in their entireties for all purposes.

The recombinant ACE2 polypeptides described in this disclosure have amino acid residue substitutions that result in substantially increased binding affinity for the spike RBD compared to wild-type ACE2, in some instances 170 fold greater binding or more. In some embodiments, the recombinant ACE2 polypeptides bind strongly to spike proteins from SARS-CoV-2 variants including the UK variant and the South Africa variant. In some embodiments, the recombinant ACE2 polypeptides bind to the UK variant and South Africa variant spike proteins with a similar off rate (e.g., within a factor of 2 or within a factor of 3) to the recombinant ACE2 polypeptides bound to the non-variant spike protein. In some instances, the recombinant ACE2 polypeptides reduce SARS-CoV-2 viral titers to essentially undetectable levels in live virus neutralization assays. As described in the Examples, the recombinant ACE2 polypeptides were developed through a combination of computational alanine scanning, computational saturation mutagenesis, deep mutational scanning, and affinity maturation with the spike RBD using yeast surface display.

### II. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. The following strains of human coronaviruses are currently known: Human coronavirus 229E (HCoV-229E); Human coronavirus OC43 (HCoV-OC43); Severe acute respiratory syndrome coronavirus (SARS-CoV or SARS-CoV-1); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019.” The coronaviruses HCoV-229E, -NL63, -OC43, and -HKU1 continually circulate in the human population and cause respiratory infections in adults and children world-wide.

“Virus” is used in both the plural and singular senses. “Virion” refers to a single infectious particle.

“Spikes” are coronavirus surface proteins that are able to mediate receptor binding and membrane fusion between the virus and host cell. Spikes are homotrimers of the S protein, which has 51 and S2 domains. In addition to mediating virus entry, the spike is an important determinant of viral host range and tissue tropism and a major inducer of host immune responses. The 51 subunit of the S protein includes the receptor binding domain (RBD).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

“Fusion protein” has its normal meaning in the art, referring to a protein comprising two different polypeptide sequences, i.e. a first domain and a second domain, that are joined or linked to form a single polypeptide. The two amino acid sequences are encoded by separate nucleic acid sequences that have been joined so that they are transcribed and translated to produce a single polypeptide. The two domains can be contiguous, separated by one or more spacer, linker or hinge sequences, or separated by an additional polypeptide domain. An “Fc-fusion protein” includes an Fc domain (i.e., a monomer corresponding to an Fc homodimer). A “RBD-fusion protein” includes an RBD domain. An “ACE2-fusion protein” includes an ACE2 domain.

A “domain” of a protein refers to a region of the protein defined by an amino acid sequence and/or a functional property. Function properties include enzymatic activity and/or the ability to bind to or be bound by another protein or nonprotein entity.

A “protein dimer” has its normal meaning in the art and refers to a protein complex formed by two protein monomers, or single proteins, which are usually non-covalently bound.

### III. Polypeptides, Polynucleotides, and Compositions

Provided in this disclosure are recombinant ACE2 polypeptides, fusion proteins, and compositions thereof having improved binding affinity for the SARS-CoV-2 spike receptor binding domain (RBD) compared to wild-type ACE2. The recombinant ACE2 polypeptides are variants of the ACE2 ectodomain and have improved binding affinity for the SARS-CoV-2 spike RBD as compared to wild-type ACE2 ectodomain. In some embodiments, the recombinant ACE2 polypeptides have improved binding affinity for the spike RBD from the B.1.1.7 and/or B.1.351 SARS-CoV-2 variants as compared to wild-type ACE2 ectodomain.

In one aspect, provided are recombinant ACE2 polypeptides comprising a soluble ACE2 receptor ectodomain polypeptide comprising an amino acid sequence having at least 80% sequence identity (e.g., at least 90%, at least 95%) to SEQ ID NO: 2 or 3 and comprising at least one of the following amino acid residue substitutions: Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, Q101R, wherein the residues are numbered with reference to SEQ ID NO:1. In some instances, the recombinant ACE2 polypeptides may include at least two of these amino acid residue substitutions.

In some embodiments, the recombinant ACE2 polypeptides comprise a soluble ACE2 receptor ectodomain polypeptide comprising an amino acid sequence having at least 80% sequence identity (e.g., at least 90%) to SEQ ID NO:2, which includes an ACE2 collectrin domain (SEQ ID NO:4). In the full length ACE2 protein, the collectrin domain connects the extracellular domain of ACE2 to its transmembrane helix. The collectrin domain may stabilize the soluble extracellular part of the protein as a dimer through inter-collectrin domain contacts as well as additional C-terminal contacts between peptidase domains. In some embodiments, the collectrin domain facilitates dimerization of two ACE2 polypeptides to form a dimer. In some embodiments, recombinant ACE2 polypeptides comprising the collectrin domain have increased affinity for the spike RBD over polypeptides that do not comprise the collectrin domain, as described, for example, in Example 5 and shown in Table 5. In some embodiments, the recombinant ACE2 polypeptides comprise an amino acid sequence having at least 80% sequence identity (e.g., at least 90%) to SEQ ID NO:3, which does not include an ACE2 collectrin domain.

In some embodiments, the recombinant ACE2 polypeptide comprises a soluble ACE2 receptor ectodomain polypeptide comprising an amino acid sequence having at least 80% sequence identity (e.g., at least 90%) to SEQ ID NO: 2 or 3 and comprising amino acid residue substitutions in at least one of the following combinations:
i. K31F, N33D, H34S, and E35Q;
ii. K31F, N33D, H34A, E35Q, N49D, N51S, N53S, E57G, and N64D;
iii. T27A, K31F, N33D, H34S, E35Q, N61D, K68R, and L79P;
iv. S19P, N33S, H34V, F40L, N49D, and L100P;
v. K31F, N33D, H34S, E35Q, W69R, and Q76R;
vi. Q18R, K31F, N33D, H34S, E35Q, W69R, and Q76R;
vii. Q18R, K31F, N33D, H34S, E35Q, W69V, and Q76R;
viii. Q18R, K31F, N33D, H34S, E35Q, W69K, and Q76R;
ix. Q18R, K31F, N33D, H34S, E35Q, W69I, and Q76R;
x. T27A, H34A, N49S, V59A, N63S, K68R, E75G, N90Q, and Q103R;
xi. K31F, N33D, H34T, N53D, W69R, and E75K;
xii. S19P, K26R, T27A, H34A, S44G, and M62T;
xiii. K31F, H34I, E35Q, and N90Q;
xiv. A25V, T27A, H34A, and F40D;
xv. K31Y, W69V, L79T, and L91P;
xvi. T27Y, H34A, and N90Q;
xvii. S19P, Q42L, L79T, and N90Q;
xviii. K31F, H34I, E35Q; or
xix. H34V and N90Q,
   wherein the residues are numbered with reference to SEQ ID NO:1.

In some embodiments, the recombinant ACE2 polypeptide comprises a soluble ACE2 receptor polypeptide comprising an amino acid sequence having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO:38 or SEQ ID NO:39. In some embodiments, the recombinant ACE2 polypeptide comprises a soluble ACE2 receptor ectodomain polypeptide comprising an amino acid sequence having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 38 or SEQ ID NO:39 and comprising amino acid residue substitutions relative to SEQ ID NO:1 in one or more of the following positions: K31F, N33D, H34S, and E35Q, wherein the residues are numbered with reference to SEQ ID NO:1.

Also provided in this disclosure are recombinant ACE2 polypeptides comprising a soluble ACE2 receptor ectodomain polypeptide comprising mutations that inactivate the peptidase function of ACE2. In the body, the peptidase domain of ACE2 catalyzes the hydrolysis of angiotensin II (a vasoconstrictor peptide) into angiotensin (1-7) (a vasodilator). As such, inactivating mutations in the peptidase domain can prevent the ACE2 polypeptides from having downstream vasodilation effects when administered to a subject. For example, inactivating the peptidase function may prevent undesired interactions of the recombinant ACE2 polypeptides or compositions comprising the recombinant ACE2 polypeptides with co-administered SARS-CoV-2 therapeutics. Exemplary SARS-CoV-2 therapeutics can include drugs that cause an increase in ACE2 effects, as discussed in Cheng et al., 2020, “Organ-protective effect of angiotensin-converting enzyme 2 and its effect on the prognosis of COVID-19,” Journal of Med Virol 92:726-730. Such drugs may include vasodilators like angiotensin II receptor blockers and ACE1 inhibitors. In some instances, an inactivating mutation in the peptidase domain of the recombinant ACE2 polypeptides described herein may prevent redundant function to a co-administered vasodilator. In some aspects, the inactivating mutations do not impact affinity of the polypeptides comprising the mutations for the SARS-CoV-2 spike RBD. In some embodiments, the recombinant ACE2 polypeptides comprise amino acid residue substitutions H374N and H378N to inactivate the peptidase function. In some embodiments, the recombinant ACE2 polypeptides comprise the amino acid residue substitution H345L to inactivate the peptidase function.

In some embodiments, the recombinant ACE2 polypeptides comprise a soluble ACE2 receptor ectodomain polypeptide comprising an amino acid sequence having at least 80% sequence identity (e.g., at least 90%) to the amino acid sequence as set forth in SEQ ID NO: 2 or 3 and comprising at least one of the amino acid residue substitutions in the sequences listed in Table 3 (see Example 3) as compared to the amino acids at positions 18-105 of SEQ ID NO:1. In some instances, the recombinant ACE2 polypeptides comprise a soluble ACE2 receptor ectodomain polypeptide comprising an amino acid sequence set forth in Table 3.

In another aspect, provided herein is a fusion protein comprising a recombinant ACE2 polypeptide as described in this disclosure fused to a dimerization domain. In such fusion proteins, the recombinant ACE2 polypeptide is the first domain located in the first half of the fusion protein, and the dimerization domain is the second domain located in the second half the fusion protein. In some embodiments, the dimerization domain is not a collectrin domain.

In some embodiments, the dimerization domain is an Fc domain. The Fc domain is able to form a homodimer. In some embodiments, the dimerization domain is an Fc domain and a hinge domain. In some embodiments, the fusion protein comprises an Fc domain (or an Fc domain and a hinge domain) and a recombinant ACE2 polypeptide that does not comprise a collectrin domain (amino acids 615-740). In some embodiments, the fusion protein comprises an Fc domain (or an Fc domain and a hinge domain) and a recombinant ACE2 polypeptide that does comprise a collectrin domain (amino acids 615-740). Any desired hinge and/or Fc domain may be used. In some embodiments, a human Fc sequence or human Fc and hinge sequence is used. In some embodiments, an Fc sequence or Fc and hinge sequences from a nonhuman primate is used. In some embodiments, an Fc sequence is used with a heterologous hinge sequence (e.g., a human Fc sequence with a nonhuman primate hinge sequence or a nonhuman primate Fc sequence with a human hinge sequence). In some embodiments, the Fc sequence and/or hinge domain used is chosen based on desired immune effector functions. For example, a human IgG4 Fc domain, alone or together with a human or nonhuman primate hinge sequence, may be used to reduce or avoid immune activation relative to a human IgG1 Fc domain. The Fc domain may also contain mutations that reduce antibody-dependent cell-mediated cytotoxicity (ADCC) and/or other innate immune effects. Aspects of IgG Fc based therapeutics design are described in Wang et al., 2018, “IgG Fc engineering to modulate antibody effector functions,” Protein & Cell 9:63-73. In some embodiments, the Fc domain is a human IgG1 Fc domain with the amino acid sequence set forth in SEQ ID NO:5, alone or together with a human IgG1 hinge domain (SEQ ID NO:6). The amino acid sequence of a human IgG1 Fc domain together with a human IgG1 hinge domain is set forth in SEQ ID NO:7. In some embodiments, the dimerization domain comprises SEQ ID NO:7. In some embodiments, the dimerization domain comprises SEQ ID NO:40. In some embodiments, the fusion protein comprises an amino acid sequence having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to any one of SEQ ID NOs:77-81.

In some embodiments, a dimerization domain other than an Fc domain is used. There are a wide array of protein dimerization domains known in the art, including commercially available constructs that can be used to express fusion proteins (e.g., iDimerize® system, Takara Bio USA).

In some embodiments, the recombinant ACE2 polypeptide is fused to the dimerization domain via a peptide linker. A linker sequence may increase the range of orientations that may be adopted by the domains of the fusion protein. The peptide linker may be, for example, 5 to 60 or more amino acids in length (e.g., 5 aa, 10 aa, 15 aa, 25 aa, 35 aa, 40 aa, 45 aa, 50 aa, 55 aa, or 60 aa). The linker sequence may be optimized to produce desired effects in the recombinant ACE2 polypeptide. Aspects of linker design and considerations are described, for example, in Koerber et al., 2015, “An improved single-chain Fab platform for efficient display and recombinant expression,” J Mol Biol 427(2):576-586, Chen, X. et al., Adv Drug Deliv Rev. 2013 Oct. 15; 65(10): 1357-1369, and Klein, J. S. et al. 2014 Protein Eng. Des. Sel. 27(10):325-330. Depending on length, linker sequence may have various conformations in secondary structure, such as helical, β-strand, coil/bend, and turns. In some instances, a linker sequence may have an extended conformation and function as an independent domain that does not interact with the adjacent protein domains. Linker sequences may be flexible or rigid. Flexible linkers provide a certain degree of movement or interaction between the polypeptide domains and are generally rich in small or polar amino acids such as Gly and Ser. A rigid linker can be used to keep a fixed distance between the domains and to help maintain their independent functions. In some embodiments, the linker comprises SEQ ID NO:8. In some embodiments, the linker comprises SEQ ID NO:9. In some embodiments, the linker comprises SEQ ID NO:10 (GSSGGGGSGGGGSGGGGSGGGG). In some embodiments, the linker comprises SEQ ID NO:11 (GSSGGGGSGGGGSGGGG). In some embodiments, the linker comprises SEQ ID NO:12 (GSSGGGGSGGGG). In some embodiments, the linker comprises SEQ ID NO:13 (CSGGGGSGGGG). Additional exemplary peptide linkers include, but are not limited to, peptide linkers comprising SEQ ID NO:14 (SGSETPGTSESATPE), SEQ ID NO:15 (SGSETPGTSESATPES), SEQ ID NO:16 ((GGGGS)₃), SEQ ID NO:17 ((GGGGS)₁₀), SEQ ID NO:18 (A(EAAAK)₃A), SEQ ID NO:19 (A(EAAAK)₁₀A), or the AviTag™ linker (GGGGS; SEQ ID NO:20).

Any of the polypeptides or proteins described herein can further comprise a detectable moiety, for example, a fluorescent protein or fragment thereof. Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al. “Fluorescent Proteins and Their Applications in Imaging Living Cells and Tissues,” Physiological Reviews 90(3): 1103-1163 (2010); and Specht et al., “A Critical and Comparative Review of Fluorescent Tools for Live-Cell Imaging,” Annual Review of Physiology 79: 93-117 (2017).

Any of the polypeptides or proteins described herein can further comprise a domain or sequence useful for protein isolation. In some embodiments, the polypeptides comprise an affinity tag, for example an AviTag™ (SEQ ID NO:21), a Myc tag (SEQ ID NO:22), a polyhistidine tag (e.g., 8×His tag (SEQ ID NO:23)), an albumin-binding protein, an alkaline phosphatase, an AU1 epitope, an AU5 epitope, a biotin-carboxy carrier protein (BCCP), or a FLAG epitope, to name a few. In some embodiments, the affinity tags are useful for protein isolation. See, Kimple et al. “Overview of Affinity Tags for Protein Purification,” Curr. Protoc. Protein Sci. 73: Unit-9.9 (2013). In some embodiments, the polypeptides or proteins described herein comprise a signal sequence useful for protein isolation, for example a mutated Interleukin-2 signal peptide sequence (SEQ ID NO:24), which promotes secretion and facilitates protein isolation. See, for example, Low et al. “Optimisation of signal peptide for recombinant protein secretion in bacterial hosts,” Applied Microbiology and Biotechnology 97:3811-3826 (2013). In some embodiments, the polypeptides or proteins described herein comprise a protease recognition site, for example, TEV protease cut site (SEQ ID NO:25). Such protease recognition sites may be useful for, among other things, allowing removal of a signal peptide or affinity purification tag following protein isolation.

In some embodiments, the recombinant ACE2 polypeptides include substitutions that improve binding or other properties. For example, one or more cysteine substitutions, or substitutions with noncanonical amino acids containing long side-chain thiols, may be introduced into the polypeptides that can form disulfide bonds between two polypeptides that have interacted to form a dimer. In some embodiments, the substitutions improve polypeptide stability. For example, the polypeptides may comprise substitution of the tryptophan residue at position 69 with reference to SEQ ID NO:1 with a valine residue, a lysine residue, or an isoleucine residue.

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et al. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

In another aspect, provided herein are compositions comprising a dimer of recombinant ACE2 polypeptides or a dimer of fusion proteins as described above. When expressed in a host cell, as discussed further below, the recombinant ACE2 polypeptide or fusion protein will form a dimer that can be isolated as a composition.

In some instances, the recombinant ACE2 polypeptides and fusion proteins provided herein have increased binding affinity for monomeric SARS-CoV-2 spike RBD relative to wild-type human ACE2 protein ectodomain. As used herein, binding affinity of the provided recombinant ACE2 polypeptides and fusion proteins and monomeric SARS-CoV-2 spike RBD is measured as the dissociation constant “K_{D}” or apparent K_{D}. Binding affinity can be determined by a variety of methods known in the art. In some instances, bio-layer inferometry can be used to measure binding affinity. For example, as described in Examples 1, 2, and 5 below, the binding of ACE2 polypeptide variants to full length spike protein can be measured by bio-layer inferometry. Bio-layer interferometry (“BLI”) is an optical technique for measuring macromolecular interactions by analyzing interference patterns of white light reflected from the surface of a biosensor tip coated with an immobilized protein, with any change in the number of molecules bound to the biosensor tip (i.e. protein-protein interactions) causing a shift in the interference pattern.

In some instances, binding affinity can be measured using titrations of purified monomeric spike RBD and yeast surface expressed recombinant ACE2 polypeptides as provided herein. The dissociation constant determined using yeast surface titrations is an estimate of the apparent K_{D} rather than the actual K_{D} due to the unknown multimerization state of the ACE2 molecule on the yeast cell surface. In some embodiments, the apparent K_{D} of the provided recombinant ACE2 polypeptides can be determined as described in Examples 1 and 3 using on-yeast protein display of variants lacking the collectrin domain with titrations of monomeric spike RBD.

In some instances, binding affinity can be measured by measuring the off rate of recombinant ACE2 polypeptides bound to spike protein or spike RBD in the presence of untagged inhibitor protein, e.g., using the method described in Example 1 herein. In some embodiments, the binding affinity of a first recombinant ACE2 polypeptide for a spike protein or spike RBD measured using this method may be expressed 1) relative to the binding affinity of a second recombinant ACE2 polypeptide to the spike protein or spike RBD, or 2) relative to the binding affinity of the first recombinant ACE2 polypeptide to a different spike protein or spike RBD. For example, the binding affinity of a first recombinant ACE2 polypeptide for spike protein or spike RBD measured using this method may be expressed as within a factor of 2 (i.e., in the range of 2-fold weaker binding to 2-fold stronger binding) or within a factor of 3 (i.e., in the range of 3-fold weaker binding to 3-fold stronger binding) relative to the binding affinity of a second recombinant ACE2 polypeptide for spike protein or spike RBD or relative to the binding affinity of the first recombinant ACE2 polypeptide for a different spike protein or spike RBD. In some embodiments, a recombinant ACE2 polypeptide that has a binding affinity for spike protein or spike RBD measured using this method with a factor of 2 or within a factor of 3 relative to the binding affinity of the recombinant ACE2 polypeptide for a different spike protein or spike RBD may be said to have “similar binding” or to bind with a “similar off rate” to the different spike proteins or spike RBDs.

Other methods of measuring binding affinity include ELISA, surface plasmon resonance, or kinetic exclusion assays (Kinexa®). The K_{D} range in which measurements are accurate for different analytical methods may vary. For example, in some instances, as described in Examples 1, 2, and 5 below, the binding of ACE2 polypeptide variants that comprise the collectrin domain to full length spike protein may be too tight to be accurately measured by bio-layer inferometry (see Example 5 and Table 5). However, the apparent K_{D} can be measured for these variants using yeast surface titrations as described in Examples 1 and 3. One of skill in the art will appreciate that, within the accurate range, these methods will result in similar binding affinity measurements or similar trends in relative binding affinities for the various ACE2 polypeptides and fusion proteins described herein as compared to wild-type ACE2 and/or other ACE2 polypeptide variants.

In some instances, binding affinity for the recombinant ACE2 polypeptides and fusion proteins provided herein with monomeric SARS-CoV-2 spike RBD may be measured as an apparent K_{D} of less than 10 nM (for example, less than 9 nM, less than 7 nM, less than 5 nM, less than 4 nM, less than 3 nM, less than 2 nM, less than 1 nM, less than 0.5 nM, less than 0.25 nM, or less than 0.1 nM). Relative to wild-type human ACE2 protein ectodomain, the provided polypeptides and fusion proteins may have between 30-fold and 180-fold higher affinity for monomeric spike RBD (for example, between 40-fold and 160-fold, between 60-fold and 140-fold, between 80-fold and 120-fold, between 30-fold and 60-fold, between 150-fold and 180-fold, greater than 50-fold, greater than 100-fold, greater than 150-fold). In some instances, the provided polypeptides and fusion proteins may have greater than 180-fold higher affinity for monomeric SARS-CoV-2 spike RBD as compared to wild-type human ACE2 protein ectodomain. In one embodiment, a recombinant ACE2 fusion protein may have a binding affinity (apparent K_{D}) for monomeric spike RBD of 0.4 nM (51-fold higher than wild-type human ACE2 protein ectodomain; see e.g., variant 310 in Examples). In another embodiment, a recombinant ACE2 fusion protein may have a binding affinity (apparent K_{D}) for monomeric spike RBD of 0.64 nM (32-fold higher than wild-type human ACE2 protein ectodomain; see e.g., variant 311 in Examples). In one embodiment, a recombinant ACE2 fusion protein may have a binding affinity (apparent K_{D}) for monomeric spike RBD of 1.71 nM (12-fold higher than wild-type human ACE2 protein ectodomain; see e.g., variant 293 in Examples). In another embodiment, a recombinant ACE2 fusion protein may have a binding affinity (apparent K_{D}) for monomeric spike RBD of 0.52 nM (39-fold higher than wild-type human ACE2 protein ectodomain; see e.g., variant 313 in Examples). The wild-type human ACE2 protein ectodomain to which the binding affinity of the recombinant ACE2 polypeptides and fusion proteins is compared can have the amino acid sequence of SEQ ID NO: 2 or 3.

In some embodiments, the recombinant ACE2 polypeptides and fusion proteins provided herein show similar binding between 1) spike proteins or spike RBD from non-variant SARS-CoV-2 virus and 2) spike proteins or spike RBD from variant SARS-CoV-2 viruses (e.g., the B.1.1.7 UK variant and/or the B.1.351 South Africa variant). In some embodiments, the recombinant ACE2 polypeptides bind to the UK variant and South Africa SARS-CoV-2 variant spike proteins with a similar off rate (i.e., within a factor of 3 or within a factor of 2) to the recombinant ACE2 polypeptides bound to non-variant SARS-CoV-2 spike protein (see, e.g., Example 9 herein).

In some instances, the recombinant ACE2 polypeptides and fusion proteins provided herein have increased efficacy in neutralizing SARS-CoV-2 virus compared to wild-type human ACE2 ectodomain. For example, the recombinant ACE2 polypeptides and fusion proteins provided herein can reduce the ability of viral particles having surface expressed SARS-CoV-2 spike protein from entering mammalian eukaryotic cells expressing ACE2, as described, for example, in Example 6 below. A measure of the efficacy of virus neutralization can be reflected by the half maximal inhibitory concentration (IC50), which indicates how much of the recombinant ACE2 polypeptide or fusion protein is needed to inhibit a biological process (i.e. viral infection) by half, thus providing a measure of potency of the recombinant ACE2 polypeptide or fusion protein. In some instances, the efficacy of the recombinant ACE2 polypeptides and fusion proteins provided herein in neutralizing spike pseudotyped lentivirus may be measured as an IC50 of less than 100 ng/mL (for example, less than 90 ng/mL, less than 80 ng/mL, less than 70 ng/mL, less than 60 ng/mL, less than 50 ng/mL, less than 45 ng/mL, less than 40 ng/mL, less than 35 ng/mL, or less than 30 ng/mL). Relative to wild-type human ACE2 protein ectodomain in the ACE2(740)-Fc format, the provided polypeptides and fusion proteins may have between 10-fold and 26-fold higher neutralization efficacy for spike pseudotyped lentivirus (for example, between 12-fold and 15-fold, between 16-fold and 20-fold, between 21-fold and 26-fold, greater than 12-fold, greater than 15-fold, greater than 18-fold, greater than 21-fold, or greater than 25-fold). In some instances, the provided recombinant ACE2 polypeptides and fusion proteins may have greater than 25-fold higher neutralization efficacy for SARS-CoV-2 virus compared to wild-type human ACE2 protein ectodomain. In one embodiment, a recombinant ACE2 fusion protein may have an IC50 value for spike pseudotyped lentivirus of approximately 58 ng/mL (12-fold higher than wild-type human ACE2 protein ectodomain; see e.g., variant 310 in Examples). In one embodiment, a recombinant ACE2 fusion protein may have an IC50 value for spike pseudotyped lentivirus of approximately 55 ng/mL (13-fold higher than wild-type human ACE2 protein ectodomain; see e.g., variant 311 in Examples). In one embodiment, a recombinant ACE2 fusion protein may have an IC50 value for spike pseudotyped lentivirus of approximately 36 ng/mL (20-fold higher than wild-type human ACE2 protein ectodomain; see e.g., variant 293 in Examples). In one embodiment, a recombinant ACE2 fusion protein may have an IC50 value for spike pseudotyped lentivirus of approximately 28 ng/mL (25-fold higher than wild-type human ACE2 protein ectodomain; see e.g., variant 313 in Examples). The wild-type human ACE2 protein ectodomain to which the virus neutralization of the recombinant ACE2 polypeptides and fusion proteins is compared can have the amino acid sequence of SEQ ID NO: 2 or 3.

In some instances, the recombinant ACE2 polypeptides, fusion proteins, and compositions provided herein may also bind to the RBD of spike proteins of coronaviruses other that SARS-CoV-2. In some instances, the recombinant ACE2 polypeptides, fusion proteins, and compositions described herein can bind to the spike protein of SARS-CoV-1 virus as well as other coronaviruses that have sufficient homology to the SARS-CoV-2 spike protein in terms of amino acid residue sequence or tertiary structure. For example, as described in Example 6 and shown in Table 8, the recombinant ACE2 polypeptides and fusion proteins are able to bind the RBD domain from the SARS-CoV-1 virus and the RBD domain from the human coronavirus NL63 (HcoV-NL63). In some instances, the spike RBD of the coronavirus has sufficient homology to the SARS-CoV-2 spike RBD in terms of amino acid residue sequence or tertiary structure to have high affinity binding to the recombinant ACE2 polypeptide, fusion protein, or compositions of the present disclosure.

Recombinant nucleic acids encoding any of the polypeptides or proteins described herein are also provided. For example, provided is a recombinant nucleic acid encoding a polypeptide comprising a soluble ACE2 receptor ectodomain polypeptide that has at least 80%, for example, at least about 85%, 88%, 90%, 91%, 92%, 93%, 94%, 95%, 96%, 97%, 98% or 99%, identity to any one of SEQ ID NOs 2-3 and comprising at least one of the following amino acid residue substitutions: Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, Q101R, wherein the residues are numbered with reference to SEQ ID NO:1.

In some embodiments, the recombinant nucleic acid encodes a polypeptide comprising a soluble ACE2 receptor ectodomain polypeptide that has at least 80%, identity (e.g., at least 90%) to SEQ ID NO: 2 or 3 and comprising amino acid residue substitutions in at least one of the following combinations:
i. K31F, N33D, H34S, and E35Q;
ii. K31F, N33D, H34A, E35Q, N49D, N51S, N53S, E57G, and N64D;
iii. T27A, K31F, N33D, H34S, E35Q, N61D, K68R, and L79P;
iv. S19P, N33S, H34V, F40L, N49D, and L100P;
v. K31F, N33D, H34S, E35Q, W69R, and Q76R;
vi. Q18R, K31F, N33D, H34S, E35Q, W69R, and Q76R;
vii. Q18R, K31F, N33D, H34S, E35Q, W69V, and Q76R;
viii. Q18R, K31F, N33D, H34S, E35Q, W69K, and Q76R;
ix. Q18R, K31F, N33D, H34S, E35Q, W69I, and Q76R;
x. T27A, H34A, N49S, V59A, N63S, K68R, E75G, N90Q, and Q103R;
xi. K31F, N33D, H34T, N53D, W69R, and E75K;
xii. S19P, K26R, T27A, H34A, S44G, and M62T;
xiii. K31F, H34I, E35Q, and N90Q;
xiv. A25V, T27A, H34A, and F40D;
xv. K31Y, W69V, L79T, and L91P;
xvi. T27Y, H34A, and N90Q;
xvii. S19P, Q42L, L79T, and N90Q;
xviii. K31F, H34I, E35Q; or
xix. H34V and N90Q,
   wherein the residues are numbered with reference to SEQ ID NO:1.

In some embodiments, the recombinant nucleic acid encodes a polypeptide comprising a soluble ACE2 receptor polypeptide having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to the amino acid sequence as set forth in SEQ ID NO:38 or SEQ ID NO:39. In some embodiments, the recombinant nucleic acid encodes a polypeptide comprising a soluble ACE2 receptor ectodomain polypeptide having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to the amino acid sequence as set forth in SEQ ID NO:38 or SEQ ID NO:39 and comprising amino acid residue substitutions relative to SEQ ID NO:1 in one or more of the following positions: K31F, N33D, H34S, and E35Q, wherein the residues are numbered with reference to SEQ ID NO:1.

In some embodiments, the recombinant nucleic acid encodes a polypeptide comprising a soluble ACE2 receptor ectodomain polypeptide having at least 80% sequence identity (e.g., at least 90%) to the amino acid sequence as set forth in SEQ ID NO: 2 or 3 and comprising at least one of the amino acid residue substitutions in the sequences listed in Table 3 as compared to the amino acids at positions 18-105 of SEQ ID NO:1. In some instances, the recombinant nucleic acid encodes a polypeptide comprising an amino acid sequence set forth in Table 3.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

### IV. Constructs, Vectors, and Cells

Also provided is a DNA construct comprising a promoter operably linked to a recombinant nucleic acid described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of the modified polypeptide. The cassette may additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette will include in the 5′ to 3′ direction of transcription: a transcriptional and translational initiation region (i.e., a promoter), a polynucleotide disclosed herein, and a transcriptional and translational termination region (i.e., termination region) functional in the cell or organism of interest. The promoters described herein are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid gene (See generally, Sambrook et al. Molecular Cloning: A Laboratory Manual, 4^{th} ed., Cold Spring Harbor Laboratory Press, Cold Spring Harbor, 2012). The vector, for example, can be a plasmid.

There are numerous E. coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the art, and include CHO cells, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of the polypeptides. Recombinant proteins produced in insect cells with baculovirus vectors undergo post-translational modifications similar to that of wild-type mammalian proteins.

A host cell comprising a nucleic acid, a DNA construct, or a vector described herein is also provided. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, Ausubel, and Berger (all supra), as well as Freshney (1994) Culture of Animal Cells, a Manual of Basic Technique, 3^{rd} Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell, or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a recombinant protein described herein, into a host cell.

Any of the proteins described herein can be purified or isolated from a host cell or population of host cells. For example, a recombinant nucleic acid encoding any of the proteins described herein can be introduced into a host cell under conditions that allow expression of the protein. In some embodiments, the recombinant nucleic acid is codon-optimized for expression. After expression in the host cell, the recombinant protein can be isolated or purified using purification methods known in the art. In some embodiments, a recombinant nucleic acid encoding a recombinant ACE2 polypeptide can be introduced into a host cell under conditions that allow expression thereof, with the expressed polypeptide forming a protein dimer. In some embodiments, a recombinant nucleic acid encoding a fusion protein comprising a recombinant ACE2 polypeptide and a dimerization domain can be introduced into a host cell under conditions that allow expression of the fusion protein, with the expressed polypeptide forming a protein dimer. After expression in the host cell, the protein dimer can be isolated or purified using purification methods known in the art. In some embodiments, the fusion protein is isolated as a monomer and allowed to dimerize in vitro.

Following expression, the recombinant ACE2 polypeptides can be isolated. Proteins can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, N.Y. The degree of purification necessary varies depending on the desired use. In some instances, no purification of the expressed antibody or fragments thereof is necessary.

One method of producing recombinant ACE2 polypeptides is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonyl) chemistry (Applied Biosystems, Inc.; Foster City, Calif.). A protein provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form a fusion protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Methods for determining the yield or purity of a purified protein are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

An “isolated” or “purified” polypeptide or protein is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the invention or its biologically active portion is recombinantly produced, optimally culture medium represents less than about 30%, 20%, 10%, 5%, 1%, 0.5%, or 0.1% (by concentration) of chemical precursors or non-protein-of-interest chemicals.

### V. Pharmaceutical Compositions and Formulations

The recombinant ACE2 polypeptides described herein are suitable for administration in vitro or in vivo. Compositions comprising a recombinant protein of the present disclosure and a pharmaceutically acceptable carrier (excipient) are provided. A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The pharmaceutical compositions may further comprise a diluent, solubilizer, emulsifier, preservative, and/or adjuvant to be used with the methods disclosed herein. Such pharmaceutical compositions can be used in a subject that would benefit from administration of any of the recombinant ACE2 polypeptides or fusion proteins described herein, for example, a subject infected with a SARS-CoV-2 virus, a subject having symptoms suggestive of a SARS-CoV-2 infection, a subject exposed to a SARS-CoV-2 virus, and/or a subject at risk of exposure to a SARS-CoV-2 virus.

Suitable carriers and their formulations are described in Remington: The Science and Practice of Pharmacy, 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically-acceptable salt to render the formulation isotonic. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the recombinant ACE2 polypeptides described herein.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a recombinant ACE2 protein or fusion protein disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising a recombinant ACE2 protein or fusion protein disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions or suspensions, including saline and buffered media) comprising a recombinant ACE2 protein or fusion protein in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product that can then be delivered via a depot injection. In certain embodiments, hyaluronic acid can also be used, and can have the effect of promoting sustained duration in the circulation.

In certain embodiments, the compositions can be selected for inhalation or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. A low pH vehicle may be useful for formulating the recombinant ACE2 polypeptide and fusion protein of this disclosure as the recombinant ACE2 polypeptides may be pH sensitive and bind the spike RBD 2-5 times more tightly at pH 6.0 as compared to at pH 7.4, as described in U.S. provisional patent application 63/022,789 and Lui, I., et al. bioRxiv, doi.org/10.1101/2020.05.21.109157, published May 21, 2020, both of which are incorporated herein in their entireties for all purposes.

In certain embodiments, a pharmaceutical composition can comprise a therapeutically effective amount of a recombinant ACE2 polypeptide or fusion protein in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a recombinant ACE2 polypeptide or fusion protein in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E. et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15: 167-277; and Langer (1982) Chem Tech 12:98-105), ethylene vinyl acetate (Hsu and Langer (1985) J Biomed Materials Res 19(4):445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In still another aspect, unit dose forms comprising a recombinant ACE2 polypeptide as described in this disclosure are provided. A unit dose form can be formulated for administration according to any of the routes described in this disclosure. In one example, the unit dose form is formulated for intravenous or intraperitoneal administration. In still another aspect, pharmaceutical packages comprising unit dose forms of a recombinant ACE2 polypeptide are provided.

The recombinant ACE2 polypeptides and fusion proteins disclosed herein are ideally suited for the preparation of a kit. In some embodiments, kits are provided for carrying out any of the methods described herein. The kits of this disclosure may comprise a carrier container being compartmentalized to receive in close confinement one or more containers such as vials, tubes, syringes, and the like, each of the containers comprising one of the separate elements to be used in the method.

A recombinant ACE2 polypeptide or fusion protein as described in this disclosure for use in treating a subject may be delivered in a pharmaceutical package or kit to doctors and subjects. Such packaging is intended to improve patient convenience and compliance with the treatment plan. Typically the packaging comprises paper (cardboard) or plastic. In some embodiments, the kit or pharmaceutical package further comprises instructions for use (e.g., for administering according to a method as described herein).

In one embodiment, the kit or pharmaceutical package comprises a recombinant ACE2 polypeptide or fusion protein in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### VI. Methods of Treatment

As described herein, the present disclosure provides a method of treating a subject, comprising administering to the subject a therapeutically effective amount of a recombinant ACE2 polypeptide or fusion protein of the present disclosure. As demonstrated in the Examples below, the recombinant ACE2 polypeptides bind the spike RBD more effectively than endogenous ACE2, preventing the SARS-CoV-2 virus from interacting with endogenous ACE2 on cellular surfaces, entering cells, and propagating. In some embodiments, the subject has or is suspected to have a SARS-CoV-2 virus infection. In some embodiments, the subject has symptoms indicative of a SARS-CoV-2 infection. In some embodiments, the subject is diagnosed as having a SARS-CoV-2 virus infection. In some embodiments, the subject has been diagnosed with COVID-19.

As used herein, the term “subject” means a mammalian subject. Exemplary subjects include, but are not limited to humans, monkeys, dogs, cats, mice, rats, cows, pigs, birds, horses, camels, goats, and sheep.

SARS-CoV-2 infection in a subject can be detected by various assays performed on a biological sample from the subject. The biological sample may be from a throat swab, a nasopharyngeal swab, sputum or tracheal aspirate, urine, feces, or blood. In some instances, nucleic acids are isolated from the biological sample and tested for the presences of viral genomic sequences. In some embodiments, PCR is performed to detect SARS-CoV-2 nucleic acids from the biological sample. In some embodiments, a subject may have antibodies that selectively bind to SAR-CoV-2 proteins, e.g., SARS-CoV-2 spike protein. Antibodies can be detected in a blood sample from the subject by immunoassay (e.g., lateral flow assay or ELISA). In some embodiments, SARS-CoV-2 is detected using a proximity-based binding assay for detection of virus and/or anti-virus antibodies, as described in Elledge et al., 2021, “Engineering luminescent biosensors for point-of-care SARS-CoV-2 antibody detection,” Nat. Biotech., doi:10.1038/s41587-021-00878-8, and U.S. Provisional Patent Application Nos. 63/022,789, 63/056,509, and 63/067,273 and the international PCT applications claiming priority thereto that are filed concurrently with the instant application, each of which is incorporated by reference in its entirety herein.

In some embodiments, the subject to be treated may display one or more symptoms indicative of SARS-CoV-2 infection (i.e. of COVID-19). Such symptoms include, but are not limited to, any of a new loss of taste or smell, myalgia, fatigue, shortness of breath or difficulty breathing, fever, and/or cough. Symptoms may also include pharyngitis, headache, productive cough (i.e. a cough that produces mucus or phlegm), gastrointestinal symptoms (e.g., diarrhea, nausea, vomiting, or abdominal pain), hemoptysis, chest pressure or pain, confusion, cyanosis, and/or chills. In some embodiments, the patient has at least two symptoms selected from the group consisting of a new loss of taste or smell, shortness of breath or difficulty breathing, fever, cough, chills, or muscle aches. In some embodiments, the subject to be treated may have a blood oxygen level reading of 94 or less, e.g., as determined by an oximeter. In some embodiments, the subject may have radiographic evidence of pulmonary infiltrates. In some embodiments, the subject may have been receiving standard support care and may continue to receive such care during treatment with the recombinant ACE2 polypeptide compositions provided herein. In some instances, the subject is also being treated with standard care, such as being administered oxygen, fluids, and/or other therapeutic procedures or agents.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., a SARS-CoV-2 virus infection and/or COVID-19, lessening in the severity or progression, or curing thereof. Treating or treatment also encompass prophylactic treatments that reduce the incidence of a disease or disorder in a subject and/or reduce the incidence or reduce severity of a symptom thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying, preventing increases in, or decreasing viral load. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a SARS-CoV-2 virus infection in a subject by administering a recombinant ACE2 polypeptide or fusion protein composition as described in this disclosure is considered to be a treatment if there is a 10% reduction in one or more symptoms of the SARS-CoV-2 virus infection in a subject as compared to a control. Thus the reduction can be a 10%, 20%, 30%, 40%, 50%, 60%, 70%, 80%, 90%, 100%, or any percent reduction in between 10% and 100% as compared to native or control levels. In some embodiments, recombinant ACE2 polypeptide formulations are administered to the subject until the subject exhibits amelioration of at least one symptom of SARS-CoV-2 infection and/or is demonstrated to have a sustained decrease in viral load, e.g., as measured by immunoassay and/or quantitative amplification method, including PCR or sequencing. In some instances, the recombinant ACE2 polypeptide formulation is administered to the subject until viral load is undetectable, i.e. below the level of detection, such that no SARS-CoV-2 RNA copies can be detected by the assay methodology employed. In some instances, the subject exhibits undetectable viral load 1-4 weeks, 2-4 weeks, 2-12 weeks, 4-12 weeks, or 12-24 weeks after last administration of the formulation. It is understood that treatment does not necessarily refer to a cure or complete ablation of the disease, condition, or symptoms of the disease or condition. Exemplary dosing regimens for the recombinant ACE2 polypeptide or fusion protein compositions described herein include those described in Zoufaly, et al., 2020, “Human recombinant soluble ACE2 in severe COVID-19,” Lancet Respir. Med. 8(11):1154-1158, doi:10.1016/S2213-2600(20)30418-5 and press release from APEIRON Biologics AG (Vienna, Austria), 2020, “APEIRON's APN01 shows clinical benefits for severely ill COVID-19 patients in phase 2 trial,” available at apeiron-biologics.com/apeirons-apn01-shows-clinical-benefits-for-severely-ill-covid-19-patients-in-phase-2-trial/.

The recombinant ACE2 polypeptides described herein can also be used as a prophylactic therapy for SARS-CoV-2 virus infection, such that the provided recombinant polypeptides are administered to high-risk subjects in a therapeutically effective amount in order to lessen the likelihood and/or severity of SARS-CoV-2 virus infection. When administered prophylactically, the proteins, complexes, and compositions are administered prior to the onset of symptoms of an infection. Prophylactic administration may prevent a SARS-CoV-2 exposure from progressing to a SARS-CoV-2 infection or prevent a SARS-CoV-2 infection from progressing to COVID-19 (i.e. presentation of symptoms). In some embodiments, the subject has been exposed to a SARS-CoV-2 virus. In some embodiments, the subject is at risk of exposure to a SARS-CoV-2 virus. For example, the subject may be a healthcare worker who has been exposed to a human patient with a suspected or confirmed SARS-CoV-2 virus infection. As another example, the subject may be identified through contact tracing efforts as having come into physical contact with a human having a confirmed SARS-CoV-2 virus infection. In some embodiments, the subject is administered a recombinant ACE2 polypeptide as described herein within 1, 2, 3, 4, or 5 days from exposure or suspected exposure to SARS-CoV-2. In some embodiments, the subject is administered a recombinant ACE2 polypeptide as described herein with 1, 2, 3, 4, or 5 days from identification of the subject as having a high risk of SARS-CoV-2 infection.

As discussed above, the recombinant ACE2 polypeptides, fusion proteins, and compositions provided herein may also bind to the RBD of spike proteins of coronaviruses other than SARS-CoV-2. Thus, also provided herein are methods of treating a subject that has or is suspected to have a coronavirus infection other than a SARS-CoV-2 virus infection, the method comprising administering to the subject a therapeutically effective amount of a recombinant ACE2 polypeptide, fusion protein, or composition of the present disclosure. In some embodiments, the subject has symptoms indicative of a coronavirus infection other than a SARS-CoV-2 virus infection. In some embodiments, the subject is diagnosed as having a coronavirus infection infection other than a SARS-CoV-2 virus infection. The recombinant ACE2 polypeptide, fusion protein, or compositions may also be administered prophylactically to a subject at risk of developing a coronavirus infection other than a SARS-CoV-2 virus infection. In some instances, the coronavirus infection is a SARS-CoV-1 virus infection. In some instances, the coronavirus infection is a human coronavirus NL63 infection. In some instances, the coronavirus infection is by a coronavirus having a spike RBD with sufficient homology to the SARS-CoV-2 spike RBD in terms of amino acid residue sequence or tertiary structure to have high affinity binding to the recombinant ACE2 polypeptide, fusion protein, or compositions of the present disclosure.

In some embodiments, the subject is administered a recombinant ACE2 polypeptide or fusion protein composition as described herein within 1, 2, 3, 4, or 5 days from the onset of symptoms or within 1, 2, 3, 4, or 5 days from testing positive for SARS-CoV-2 infection or other coronavirus infection. In some embodiments, the subject is administered a recombinant ACE2 polypeptide or fusion protein composition as described herein within 1 or 2 days of hospitalization with one or more symptoms indicative of SARS-Cov-2 infection or other coronavirus infection.

“Administering” or “administration of” a composition to a subject (and grammatical equivalents of this phrase), as used herein, refer to the act of injecting or otherwise physically delivering a substance as it exists outside the body (e.g., a recombinant ACE2 polypeptide or fusion protein composition provided herein, a nucleotide construct encoding same, or a pharmaceutical composition comprising a recombinant ACE2 polypeptide or fusion protein) into a subject. Administration can be via enteral or parenteral routes. In some embodiments, administration is by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, delayed, or reduced in severity, administration of the substance typically occurs before the onset of the disease or symptoms thereof. Administration refers to direct administration, which may be administration to a subject by a medical professional or may be self-administration, and/or indirect administration, which may be the act of prescribing a composition. For example, a physician who instructs a subject to self-administer a composition and/or provides a subject with a prescription for a composition is administering the composition to the subject.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing polypeptides are well known to those of skill in the art. Generally, such systems should utilize components that will not significantly impair the biological properties of the polypeptides, such as the capacity to bind the spike RBD (see, for example, Remington's Pharmaceutical Sciences, 18th edition, 1990, Mack Publishing). Those of skill in the art can readily determine the various parameters and conditions for producing polypeptide injectates or infusates without resorting to undue experimentation. Administration can be achieved by, e.g., topical administration, local administration, injection, by means of an implant.

As used herein, the term “therapeutically effective amount” refers to an amount of recombinant ACE2 polypeptide or fusion protein composition as described herein that, when administered to a subject, is effective to achieve an intended purpose, e.g., to reduce viral load or prevent viral load from increasing, to reduce or ameliorate at least one symptom of SARS-CoV-2 infection or other coronavirus infection, and/or otherwise reduce the length of time that a patient experiences a symptom of SARS-CoV-2 infection or other coronavirus infection, or extend the length of time before a symptom may recur. A therapeutically effective amount is not, however, a dosage so large as to cause adverse side effects, such as hyperviscosity syndromes, pulmonary edema, congestive heart failure, and the like. A therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. Human doses can be extrapolated from animal studies. Generally, the dosage required to provide an effective amount of a formulation, which can be adjusted by one skilled in the art, will vary depending on the age, health, physical condition, weight, type and extent of the disease or disorder of the recipient, frequency of treatment, the nature of concurrent therapy (if any), the method of administration, and the nature and scope of the desired effect(s) (Nies et ah, Chapter 3 In: Goodman & Gilman's The Pharmacological Basis of Therapeutics, 9th Ed., Hardman et ah, eds., McGraw-Hill, New York, N.Y., 1996). It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication. In some instances, a therapeutically effective amount may vary from about 0.01 mg/kg to about 50 mg/kg, preferably from about 0.1 mg/kg to about 20 mg/kg, most preferably from about 0.2 mg/kg to about 2 mg/kg, in one or more dose administrations daily, for one or several days.

In some embodiments, the recombinant ACE2 polypeptide composition is administered to the subject at least once a day, at least twice a day, or at least three times a day. In some embodiments, the recombinant ACE2 polypeptide composition is administered on consecutive days or on non-consecutive days. In some instances, the recombinant ACE2 polypeptide composition is administered to the subject for at least 1 day, at least 2 days, at least 4 days, at least 5 days, at least 6 days, at least 1 week, at least 2 weeks, at least 3 weeks, at least 1 month, at least 2 months, or at least 3 months. In some embodiments, the recombinant ACE2 polypeptide composition is administered to the subject for 2 to 5 or more days after the viral load is undetectable in order avoid “rebound” of virus replication.

A pharmaceutical preparation as described herein can comprise a therapeutically effective amount of a recombinant ACE2 polypeptide or fusion protein composition described herein. Such effective amounts can be readily determined by one of ordinary skill in the art as described above. Considerations include the effect of the administered recombinant ACE2 polypeptide, or the combinatorial effect of the recombinant ACE2 polypeptide with one or more additional active agents, if more than one agent is used in or with the pharmaceutical composition.

Suitable human doses of any of the recombinant ACE2 polypeptide described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of the recombinant ACE2 polypeptides and fusion protein compositions described herein can be determined by known pharmaceutical procedures in cell cultures or experimental animals (e.g., animal models of any of the disease states described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A recombinant ACE2 polypeptide or fusion protein composition that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects. Wild-type (WT) human recombinant ACE2 (hrACE2/APN01) was previously found to be safe in humans for the treatment of hypertension and acute respiratory distress syndrome (see Haschke et al., 2013, “Pharmacokinetics and pharmacodynamics of recombinant human angiotensin-converting enzyme 2 in healthy human subjects,” Clin Pharmacokinet 52:783-792 and Khan et al., 2017, “A pilot clinical trial of recombinant human angiotensis-converting enzyme 2 in acute respiratory distress syndrome,” Crit Care Lond Engl 21:234). The polypeptides and fusion proteins of this disclosure are expected to be similarly safe for therapeutic use.

The data obtained from the cell culture assays and animal studies can be used in formulating a range of dosage for use in humans. The dosage of a recombinant ACE2 polypeptide or fusion protein composition lies generally within a range of circulating concentrations of the recombinant ACE2 polypeptide or fusion protein compositions that includes the ED₅₀ with little or no toxicity. The dosage may vary within this range depending upon the dosage form employed and the route of administration utilized. For recombinant ACE2 polypeptides described herein, the therapeutically effective dose can be estimated initially from cell culture assays. A dose can be formulated in animal models to achieve a circulating plasma concentration range that includes the EC₅₀ (i.e., the concentration of the construct—e.g., polypeptide—that achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

In some embodiments, a recombinant ACE2 polypeptide or fusion protein composition described herein can be administered to a subject as a monotherapy. Alternatively, the recombinant ACE2 polypeptide or fusion protein composition can be administered in conjunction with other therapies for viral infection (combination therapy). For example, the composition can be administered to a subject at the same time, prior to, or after, a second therapy. In some embodiments, the recombinant ACE2 polypeptide or fusion protein composition and the one or more additional active agents are administered at the same time. Optionally, the recombinant ACE2 polypeptide or fusion protein composition can be administered first in time and the one or more additional active agents are administered second in time. In some embodiments, the one or more additional active agents are administered first in time and the recombinant ACE2 polypeptide or fusion protein composition is administered second in time. Optionally, the recombinant ACE2 polypeptide or fusion protein composition and the one or more additional agents can be administered simultaneously in the same or different routes. For example, a composition comprising the recombinant ACE2 polypeptide optionally contains one or more additional agents.

In certain embodiments, the other therapies may include administration of, for example, remdesivir, chloroquine, tenofovir, entecavir, and/or protease inhibitors (lopinavir/ritonavir). In certain embodiments, the other therapies may include administration of annexin-5, anti-PS monoclonal or polyclonal antibodies, bavituximab, and/or bind to viral glucocorticoid response elements (GREs), retinazone and RU486 or derivatives, cell entry inhibitors, uncoating inhibitors, reverse transcriptase inhibitors, integrase inhibitors, transcription inhibitors, antisense translation inhibitors, ribozyme translation inhibitors, prein processing and targeting inhibitors, protease inhibitors, assembly inhibitors, release phase inhibitors, immunosystem modulators and vaccines, including, but not limited to Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer, Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, and combinations thereof.

A recombinant ACE2 polypeptide described herein can replace or augment a previously or currently administered therapy. For example, upon treating with a recombinant ACE2 polypeptide, administration of the one or more additional active agents can cease or diminish, e.g., be administered at lower levels or dosages. In some embodiments, administration of the previous therapy can be maintained. In some embodiments, a previous therapy is maintained until the level of the recombinant ACE2 polypeptide reaches a level sufficient to provide a therapeutic effect.

Monitoring a subject (e.g., a human patient) for an improvement of a SARS-CoV-2 viral infection, as defined herein, means evaluating the subject for a change in a disease parameter, e.g., a reduction in one or more symptoms of SARS-CoV-2 virus infection exhibited by the subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a viral infection described herein.

### EXAMPLES

The following examples are offered to illustrate, but not to limit the claimed invention. Many of the following examples are further described in Glasgow et al., 2020, “Engineered ACE2 receptor traps potently neutralize SARS-CoV-2,” Proc. Nat. Acad. Sci. 117(45):28046-28055, which is hereby incorporated by reference in its entirety. Reference is made to this Glasgow et al. 2020 publication for illustration of certain experimental data as described in the instant disclosure.

### Example 1. Materials and Methods Used in Examples

The position of amino acid residue positions identified for the ACE2 polypeptides in the Examples is made with reference to SEQ ID NO:1. In the Examples, the term “ACE2” is used to refer to the recombinant wild-type and variant ACE2 ectodomain polypeptides and fusion proteins. The ACE2 ectodomain polypeptides having amino acid residues 18-614 relative to SEQ ID NO:1 (or variants thereof) are generally referred to as “ACE2(614)”, and the ACE2 ectodomain polypeptides having amino acid residues 18-740 relative to SEQ ID NO:1 (or variants thereof) are generally referred to as “ACE2(740)”.

### A. Structural Modeling and Computational Protein Design

The 2019.38 release of Rosetta 3.12 (Git SHA1 hash number: 2019.38.post.dev+231.master.04d3e581085 04d3e581085629b0f0c46f1e1ad9e61978e0eeb) was used for structural modeling and computational protein design.

To model ACE2-spike interactions, the 2.50 Å resolution X-ray structure of the spike receptor binding domain (RBD) complexed with the soluble extracellular domain of ACE2 (PDB 6LZG; see Wang et al., 2020, “Structural and Functional Basis of SARS-CoV-2 Entry by Using Human ACE2,” Cell 181:894-904) was used. This structure was downloaded from the PDB, relaxed with coordinate constraints on backbone and sidechain heavy atoms, and minimized in Rosetta without constraints using default options using the beta_nov16 score function (see command lines in the “Supplemental computational methods” section).

To determine which residues contribute most strongly to the ACE2-spike interaction, the Robetta Computational Interface Alanine Scanning Server (publicly available at robetta.bakerlab.org/alascansubmit.jsp; see Kortemme and Baker, 2002, “A simple physical model for binding energy hot spots in protein-protein complexes,” Proc Natl Acad Sci 99:14116-14121 and Kortemme et al., 2004, “Computational Alanine Scanning of Protein-Protein Interfaces,” Sci STKE, pl2-pl2) was used to perform a computational alanine scan on the relaxed and minimized input structure of the complex. The alanine scan identified 18 ACE2 residues in the interface, six of which had DDG(complex) values greater than 1. These “hotspots” represent amino acid side chains that are predicted to significantly destabilize the interface when mutated to alanine.

Two metrics were used to determine which hotspots could most likely be mutated to improve the ACE2-spike binding affinity: (1) the total per-residue energy as evaluated by the Rosetta score function to be the sum of all one-body and half the sum of all two-body energies for that residue, and (2) the total contribution of the residue to the interface energy, which is the sum of pairwise residue energies over all residue pairs (R1, R2) where R1 belongs to ACE2 and R2 belongs to the spike RBD. Hotspot residues that had total residue energies in the top 30% of all residues in ACE2 as well as total cross-interface interaction energies greater than 0.5 REU were classified as residues in the ACE2-spike interface to be targeted for design. These residues were H34, Q42, and K353.

For saturation mutagenesis, H34, Q42 and K353 in ACE2 were systematically computationally mutated to every other amino acid except cysteine, allowing all residues with sidechain heavy atoms in ACE2 or spike within 6 Å of the mutated position to repack (change rotameric conformation), the entire complex was minimized, and all of the pairwise interaction energies across the ACE2-spike interface and various interface metrics were recalculated. These interface metrics were: the solvent-accessible surface area buried at the interface; the change in energy when ACE2 and spike RBD are separated vs. when they are complexed; the energy of separated chains per unit interface area; the number of buried and unsatisfied hydrogen bonds at the interface; a packing statistic score for the interface; the binding energy of the interface calculated with cross-interface energy terms; a binding energy calculated using Rosetta's centroid mode and score3 score function; the number of residues at the interface; the average energy of each residue at the interface; the energy of each side of the interface; the average per-residue energy for each side of the interface; the average energy of a residue in the complex; the total number of cross-interface hydrogen bonds; and the interface energy from cross-interface hydrogen bonds. Each point mutation was modeled five times using this protocol, and the lowest of the summed cross-interface pairwise interaction energies from the five trials was used for comparison to the wild-type interface value.

Two additional sets of models were generated to select constructs for experimental testing that incorporated the most and least energetically favorable point mutations to H34 from the computational saturation mutagenesis. In these simulations, H34 was first mutated to either valine or isoleucine. Residues within 6 Å of the interface were repacked, and minimization was applied to the interface backbone and sidechain torsion angles. A flexible backbone design algorithm (Coupled Moves; see Ollikainen et al., 2015, “Coupling Protein Side-Chain and Backbone Flexibility Improves the Re-design of Protein-Ligand Specificity,” PLoS Comp Biol 11) was applied to allow neighboring ACE2 residues 30, 31, 35, and 38 to change amino acid identities while allowing ACE2 residues 29, 32, 33, 34, 36, and 37 and RBD residues 416, 417, 418, 452, 453, 455, 456, 492, 493, 494 to repack. The position of amino acid residue positions identified for RBD herein is given with reference to SEQ ID NO:26 (wild-type spike protein). Changes in the positions of backbone atoms were allowed for ACE2 residues 30-38 and RBD residues 417, 453-455, and 493. The whole complex in the lowest-energy solution for the redesigned interface was again repacked and minimized, and the final structure was scored. The lowest of the summed cross-interface pairwise interaction energies from 20 trials was used.

### B. Generation of Plasmids, Strains, and Proteins

ACE2 variants were cloned into an InvivoGen pFUSE-based vector modified to include human IgG1 hinge and Fc (SEQ ID NO:7) and biotinylation enzyme BirA (see Martinko et al., 2018, “Targeting RAS-driven human cancer cells with antibodies to upregulated and essential cell-surface proteins,” eLife 7:e31098) for mammalian expression using the Gibson method, transformed into XL10-Gold cells, and grown on low-salt LB+25 μg/ml Zeocin. The coding sequence for ACE2(18-614) (SEQ ID NO:3) or ACE2(18-740) (SEQ ID NO:2) was inserted between two SpeI sites with a 5′ mutated IL-2 signal sequence for secretion (SEQ ID NO:24) and 3′ sequences for Gly-Ser linker (SEQ ID NO:8), TEV protease cut site (SEQ ID NO:25), human IgG1 hinge and Fc (SEQ ID NO:7), and AviTag™ (Avidity) (SEQ ID NO:21). An exemplary vector sequence is SEQ ID NO:27, which includes at positions 636 to 2804 a coding sequence for an ACE2(18-740) variant polypeptide comprising the following amino acid substitutions: K31F, N33D, H34S, E35Q, and H345L. ACE2 point mutations were made in the modified pFUSE vector by stitching 5′ and 3′ fragments encoding the desired mutation by PCR and inserting the resulting coding sequence into ACE2 vectors digested with KasI and BsiWI using the Gibson assembly method.

ACE2 variants were inserted into the pCL2 vector for yeast display experiments. The coding sequence for wild-type ACE2(18-614) was inserted into NheI/BamHI-double digested pCL2 as follows: fragments encoding amino acids 18-105 and 106-614 were PCR-amplified with 25-bp Gibson overlap regions introducing a silent mutation at S105 (numbered with reference to SEQ ID NO:1) to introduce a new BamHI site, and a 3′ silent mutation to eliminate the BamHI site in pCL2. These fragments were stitched together by PCR and inserted into pCL2, transformed into XL10-Gold, and grown on LB+50 μg/ml carbenicillin. ACE2 point mutations were made in the pCL2 vector by stitching 5′ and 3′ fragments encoding the desired mutation by PCR and inserting the resulting coding sequence into pCL2 ACE2 vectors digested with NheI and BamHI using the Gibson assembly method. ACE2 variants from yeast display were amplified and cloned into the ACE2(18-740)-Fc fusion vector (modified pFUSE vector described above) in between the BlpI restriction sites on the IL-2 signal peptide and in the ACE2 coding sequence.

The SARS-CoV-2 spike RBD was cloned into the same modified pFUSE-based vector referenced above to express spike RBD-human IgG1 hinge/Fc fusion proteins (spike RBD-Fc). The RBD monomer was also cloned into a similar construct where the hinge and Fc domain was replaced with an 8×His tag (SEQ ID NO:23). A modified spike protein having the sequence set forth in SEQ ID NO:28 was also used for the BLI experiments described below. This modified spike protein sequence is modified slightly from the wild-type sequence (SEQ ID NO:26) of the first virus isolate, Wuhan-Hu-1 as described in Amanat et al., 2020, “A serological assay to detect SARS-CoV-2 serconversion in humans,” Nature Medicine 26:1033-1036 and Wu et al., 2020, “A new coronavirus associated with human respiratory disease in China,” Nature 579:265-269). A plasmid encoding the modified spike protein was obtained from the Krammer laboratory in the Department of Microbiology at Icahn School of Medicine at Mount Sinai (see labs.icahn.mssm.edu/krammerlab/covid-19/) and used to express and purify protein as described below for use in BLI experiments.

The spike RBD of human coronavirus SARS-CoV-1 having the amino acid sequence set forth in SEQ ID NO:29 was expressed from an expression plasmid having the nucleotide sequence set forth in SEQ ID NO:30 and purified as described below for use in BLI experiments.

The spike RBD of human coronavirus NL63 (HcoV-NL63) having the amino acid sequence set forth in SEQ ID NO:31 was expressed from an expression plasmid having the nucleotide sequence set forth in SEQ ID NO:32 and purified as described below for use in BLI experiments.

A cell line derived from Expi293 cells expressing an ER-localized biotin ligase (BirA) gene was grown in Expi293 media (ThermoFisher Scientific) supplemented with 100 μM biotin (GoldBio) under 8% CO₂ at 37° C. and used for all protein expressions. Transfections were carried out in 30 ml media as described in the Expi293 manual: 75 million cells at >98% viability were suspended in 25.5 ml media with biotin. Expifectamine (81 μl) and plasmid DNA (30 μg total) were mixed separately with 1.5 ml OptiMEM each, incubated at room temperature for 5 minutes, combined, and incubated for 20 minutes. The mixture was added to the cells, and after 20 hours of growth Enhancers 1 and 2 (150 μl and 1.5 ml, respectively) were added. Proteins were allowed to express for 5 days.

Expi293 cells were spun down at 3000×g for 20 minutes, and the supernatant from each culture containing protein of interest was collected, filtered through a 0.22 μm syringe filter and neutralized with 10x PBS. The supernatants were purified using a peristaltic pump and a HiTrap Protein A column (GE Healthcare). Fc-fused proteins were acid-eluted into into 1× phosphate buffered saline (PBS, 0.01 M phosphate buffer, 0.0027 M KCl and 0.137 M NaCl, Millipore Sigma P4417-100TAB), pH 7.4, from protein A columns, and then buffer-exchanged into 1×PBS, pH 7.4, using spin concentration columns (Millipore Sigma). Later ACE2(740)-Fc constructs were eluted with 50 mM Tris pH 7.2, 4 M MgCl₂ and similarly buffer exchanged. Biotinylation was quantified by denaturing proteins at 0.1 mg/ml in Lamelli buffer with 5 mM DTT for 5 minutes at 95° C., followed by addition of a molar excess of avidin and SDS-PAGE. Proteins were >95% pure and >95% biotinylated as determined by ImageJ analysis.

### C. Biophysical Characterization of ACE2 Mutants

Determination of binding affinity to spike using bio-layer interferometry (BLI): In the BLI experiments using the BioForte™ system, the biotinylated ACE2 variant was tethered to an optically transparent biosensor tip by a biotin-streptavidin interaction, and modified spike (SEQ ID NO:28) or RBD (SEQ ID NO:33) was present as the analyte in solution in the microplate. Affinity measurements were carried out at room temperature using an Octet RED96 system and streptavidin (SA)-coated biosensor tips (Pall ForteBio). Biotinylated ACE2 variants were diluted to 10 nM in PBS with 0.2% BSA and 0.05% Tween-20 (PBS-T), pH 7.4 to be used as the antigen. Antigen-bound SA-tips were washed in in PBS-T pH 7.4, separately exposed to the spike solutions at concentrations ranging from 0 to 50 nM spike in the same buffer during an association period, and then returned to the washing well during a dissociation period. The binding protocol was as follows: rinse tips in PBS-T, 60 seconds; load tips with antigen, 180 seconds; establish baseline by rinsing tips in PBS-T buffer, 180 seconds; association with analyte, 600 seconds; dissociation in baseline wells, 900 seconds. Raw data was fit to 1:1 binding curves in Octet Data Analysis HT software version 10.0 using curve fitting kinetic analysis with global fitting. The theoretical equilibrium binding signal response data (R equilibrium) were normalized by the steady-state group maximum response (Rₘₐₓ) values, and the steady state affinity was determined using the Hill equation. Non-cooperative binding kinetics were assumed. All fits to BLI data had R2 (goodness of fit)>0.90.

Measurement of off rate of receptor trap variants interacting with spike or spike RBD: The construct (receptor trap variant) being evaluated was first bound to the spike protein or RBD at 2 nM each of construct and spike. They were incubated overnight (˜16 hours) to allow for association. Then 100 nM-1000 nM (50×-500× molar excess, sufficient for re-binding to be negligible) of untagged inhibitor protein (e.g., LCB1) was added to outcompete the interaction. This is a common experimental technique for observing off rate. Samples were incubated with inhibitor for a specified amount of time, then Alpha beads were added, the samples were equilibrated with the beads for an hour, and a measurement was taken. The recorded time is the time that the complex spent in the presence of inhibitor. Observed Alpha signal corresponds to complexes that have not dissociated over the course of the incubation with inhibitor. The data are normalized to the no inhibitor condition to find the fraction still bound after the given time has passed. The data are a snapshot of the dissociation curve of the construct being tested. A value of 1 (when the data are normalized to the no inhibitor condition) means that the protein has not appreciably dissociated over the course of the experiment. A value of 0.5 would indicate that one complex half life has passed. A value of 0 would indicate that the protein has completely dissociated. For further description of the measurement technique, see Beckman Coulter, Inc. webinar presentation given by Andrew Hunt and Alex Pierson on Mar. 16, 2021 titled “Accelerating antibody discover with cell free protein synthesis and automation,” available at aiche.org/academy/webinars/accelerating-antibody-discovery-cell-free-protein-synthesis-and-automation.

Determination of stability by circular dichroism (CD) spectroscopy with thermal denaturation: CD data were collected on a Jasco J-710 spectrometer using purified ACE2 variant solutions in 1 mm quartz cuvettes. ACE2 variants were diluted in 300 μl PBS, pH 7.4 to concentrations ranging from 2 to 3 μM. Melting curves at 225 nm were measured by increasing the temperature from 25° C. to 80° C. using a rate of 1° C. per minute. CD spectra from 200 to 280 nm were measured at 25° C. and 80° C. Melting curve data were normalized using an average of the before-melt baseline as 0% and an average of the after-melt baseline as 100%, and the apparent melting temperature, T_{m,app} was determined to be the temperature at which 50% of the protein was denatured between these points. Melting was irreversible for WT ACE2.

ACE2 proteolytic activity assay: Hydrolysis of (7-methoxycoumarin-4-yl)acetyl-Ala-Pro-Lys(2,4-dinitrophenyl)-OH (Mca-APK-DNP, Enzo Life Sciences) was used to quantify ACE2 peptidase activity. 50 μl each of solutions of 0.3 nM ACE2 variants and 100 μM Mca-APK-DNP in 50 mM MES buffer, 1 M NaCl, and 10 μM ZnCl₂ were mixed in a 96-well plate. Fluorescence increase over time was monitored (320 ex./405 em.).

Library preparation: ACE2 H34V, N90Q, H34V/N90Q, and K31F/H34I/E35Q were cloned into pCL2 as N-terminal fusions to Aga2p followed by eGFP. A silent BamHI site was incorporated at G104/S105, and the BamHI site at the C-terminal linker in pCL2 was deleted for downstream library generation. 1 ng was used as template for initial error-prone PCR using 8-oxo-GTP and dPTP (TriLink Biotechnologies). Four 50 μl PCR reactions were performed on each template with increasing concentrations of mutagenic nucleotides (5 μM, 10 μM, 50 μM, and 100 μM). The 5 and 10 μM reactions were carried out using Taq polymerase in standard buffer under the following conditions: Initial denaturation at 95° C. for 30 seconds, 20 cycles of PCR (95° C. for 20 seconds, 55° C. for 20 sec, 68° C. for 45 sec), and final elongation at 68° C. for 300 sec. These reactions were loaded onto a 2% agarose gel, and the resulting bands at ˜330 bp were excised and purified. The 50 and 100 μM mutagenic PCRs were carried out similarly except with 5 cycles of PCR followed by DpnI digestion of the template for several hours at 37° C. After heat inactivation of the DpnI, 5 μl of crude PCR reaction mixture were used as template for a standard Phusion PCR and gel purified as above. To generate enough DNA for yeast transformation 150 ng from each of these 16 PCRs were used as templates for Phusion PCRs (2×50 μl reactions each). After spot-checking several reactions for the expected products by agarose gel, these reactions were pooled into 4 tubes and ethanol precipitated with 0.1 volumes of 3 M sodium acetate pH 5.2, 0.1 μg glycogen, and 3 volumes of ethanol. These were incubated several hours at room temperature followed by overnight at −80° C. and centrifuged for 20 minutes at 16,000×g. Pellets were washed with cold 70% ethanol and centrifuged again. After removing the supernatant, the pellets were air-dried and suspended in 20 μl of sterile water and centrifuged again.

Yeast transformations: Electrocompetent EBY100 were prepared by the method of Benatuil et al (see Benatuil et al, 2010, “An improved yeast transformation method for the generation of very large human antibody libraries,” Protein Eng Des Sel 23:155-159). Briefly, a stationary phase culture of EBY100 was subcultured to an OD600 of 0.3 in 200 ml YPD media and grown at 30° C. with shaking at 250 rpm for 4.5 hours. Upon reaching OD600=1.6, cells were centrifuged at 3000×g for 3 minutes, washed twice with 100 ml ice-cold MilliQ water and once with 100 ml ice-cold electroporation buffer (1 M sorbitol, 1 mM CaCl2). Cells were resuspended in 50 ml 0.1 M lithium acetate/0.01 M DTT and incubated at 30° C. for 30 minutes with shaking at 250 RPM. Cells were pelleted and washed once more with 100 ml electroporation buffer and resuspended in a minimal volume of electroporation buffer. Sublibraries prepared above were pooled into four separate electroporation cuvettes by parental ACE2 variant. A total of 30 μg of each pool was mixed with 10 μg pCL2-ACE2(18-614)BamHI-Aga2-sfGFP previously digested with NheI-HF and BamHI-HF and mixed with 400 μl electrocompetent EBY100 and incubated on ice for 5 minutes. Cells were electroporated using a Biorad Gene Pulser Xcell with an exponential pulse (2.5 kV and 25 μF), pooled, and recovered in 40 ml of 1:1 YPD:electroporation buffer at 30° C. with shaking at 250 rpm. After 2 hours the cells were centrifuged and resuspended in SDCAA and diluted up to 500 ml in SDCAA. Serial dilutions starting at 1/100 were plated on SDCAA agar plates. After 4 days at 30° C., 56 colonies were counted on a 1/100 dilution plate for a total library size of 2.8×10′.

Flow cytometry: Flow cytometry analysis of individual ACE2-expressing yeast clones was carried out using a Beckman Coulter Cytoflex flow cytometer. Approximately 50,000 cells from an overnight SGCAA culture were pelleted by centrifugation at 3,000×g for 3 minutes and washed in HyClone PBS+3% BSA (PBSA). Cells were resuspended in 100 μl PBSA, and appropriate volumes of biotinylated RBD monomer were added to avoid ligand depletion (estimating 50,000 copies of ACE2 per cell). These were incubated 2-4 hours at room temperature with rotation to reach equilibrium (see Van Deventer and Wittrup in “Monoclonal Antibodies: Methods and Protocols,” 2014, eds. Ossipow and Fischer, Meth Mol Biol 151-181), washed three times with PBSA, and stained for 20 minutes on ice with 1 μg/ml Alexa Fluor 647-conjugated streptavidin (Thermofisher Scientific S21374). After two more washes with HyClone PBS (without BSA) cells were suspended in 200 μl and analyzed. To fit yeast binding data to KDs, Alexa Fluor 647 mean fluorescence intensities were extracted from the GFP-positive population, background subtracted using secondary only controls, normalized to the highest fluorescent population, and fit to the Hill equation without cooperativity in Python.

Library screening: Cells were sorted using a BD FACS ARIA II. For sort 1, approximately 10⁸ induced library cells were washed twice with PBSA and stained with 50 nM biotinylated RBD monomer (10 ml) for 1 hour at room temperature. After washing 3× with 5 ml ice-cold PBSA, cells were incubated on ice with a 1/1000 dilution of streptavidin Alexa Fluor 647 conjugate. Cells were washed twice with 10 ml PBSA and immediately used to sort binding clones. For sort 1, the top 5% of RBD binders from 5×10⁷ cells were sorted into 3 ml SDCAA. These were pelleted at 3000×g for 3 minutes and used to inoculate 50 ml in SDCAA. After overnight growth at 30° C., cells were spun down and used to start a 50 ml, OD=1 culture in SGCAA. For sort 2, 10⁷ cells were washed and stained with 10 ml of 5 nM biotinylated RBD monomer and processed similarly to round 1. The top 0.25% of RBD binders were sorted into 3 ml SDCAA, diluted to a 5 ml culture in SDCAA, and grown at 30° C. overnight. For sorts 3.1 and 3.2, cells from sort 2 were subcultured (starting OD=1 in SGCAA) and induced at 20° C. overnight. 10⁷ cells were washed with PBSA stained with 500 pM or 200 pM RBD monomer (80 ml) for 4 hours at room temperature followed by washing and secondary staining as above. Approximately 6×10⁶ cells were analyzed and the top 1% were collected and cultured in 20 ml SDCAA. For sort 4, cells from sort 3 were subcultured as above. 5×10⁶ cells were washed in PBSA and stained with 5 ml 5 nM RBD monomer for 30 minutes at room temperature, followed by 3 washes with PBSA. Cells were resuspended in 10 ml PBSA with 20 nM H34V-ACE2(614)-Fc and incubated at room temperature for 8 hours with rotation. Cells were washed once and stained with 2 ml 1/1000 streptavidin Alexa Fluor 647 for 20 minutes, washed twice with PBSA and resuspended in 1 ml PBSA. The top 1% of cells were sorted and cultured in 20 ml SDCAA. 100 μl of a 1/100 dilution of the culture was plated on SDCAA agar to isolated individual clones for analysis. Sort 5 was performed similarly to sort 4 but with a 12-hour dissociation with 20 nM soluble H34V-ACE2(614)-Fc as a competitor. The top 0.2% of cells were collected and cultured as above.

Analysis of yeast library: To analyze sequences, plasmids were isolated from yeast using a modified version of Singh and Weil (see Singh and Weil, 2002, “A method for plasmid purification directly from yeast,” Anal Biochem 307:13-17). 5-10 ml of saturated SDCAA culture were pelleted and resuspended in 200 μl buffer P1. 10 μl of Zymolyase (Zymo Research E1004) or 50 μl Lyticase from Arthrobacter luteus (Sigma L4025-25KU) were added and the cells were incubated at 37° C. for 1-2 hours. An equal volume of buffer P2 was added and cells were incubated 10 minutes at room temperature with gentle mixing. 350 μl buffer N3 was added and the lysate was centrifuged at 16,200×g for 10 minutes. The supernatant was applied to an EconoSpin miniprep column, washed with 500 μl of buffer PB followed by 750 μl buffer PE, and eluted in 50 μl buffer EB. Library pools were transformed into XL10-Gold competent cells and plated for individual colonies on LB+50 μg/ml carbenicillin agar plates, while individual clones were amplified directly by PCR.

Virus neutralization assays: Pseudotyped reporter virus assays were conducted as previously described using spike pseudotyped lentivirus particles, i.e. lentivirus particles that express the SARS-CoV-2 spike protein as described by Crawford et al., 2020, “Protocol and Reagents for Pseudotyping Lentiviral Particles with SARS-CoV-2 Spike Protein for Neutralization Assays,” Viruses 13:513. Pseudovirus plasmids were a gift from Peter Kim's lab at Stanford University. HEK-ACE2 cells were a gift from Arun Wiita's lab at the University of California, San Francisco. Cells were cultured in D10 media (DMEM+1% Pen/Strep+10% heat-inactivated FBS). Spike-pseudovirus with a luciferase reporter gene was prepared by transfecting the pseudovirus plasmids into HEK-293T cells. After 24 hours, the transfection solution was replaced with D10 media and the virus was propagated for 48 hours before harvest and filtration of supernatants. To titer each virus batch, HEK-ACE2 were seeded at 10,000 cells and infected with two-fold dilution series of stock virus for 60 hours. Cellular expression of luciferase reporter indicating viral infection was determined using Bright-Glo™ Luciferase Assay System (Promega). For neutralization assays, virus stock was diluted to approximately 3-5×10⁵ luminescence units.

Pseudovirus neutralization assays were performed on HEK-ACE2 cells seeded at 10,000 cells/well in 40 μL of D10. To determine IC50, blocker dose series were prepared at 3× concentration in D10 media. In 96-well format, 50 μL of 3× blocker and 50 μL of virus were mixed in each well, and the virus and blocker solution was incubated for 1 hr at 37° C. After pre-incubation, 80 μL of the virus and blocker inoculum were transferred to HEK-ACE2 cells. Infection was carried out for 60 hours at 37° C., at which point intra-cellular luciferase signal was measured using the Bright-Glo™ Luciferase Assay. Neutralization was determined by normalizing the luminescent signal to the average value of the no blocker control. IC50 average values and standard deviations were calculated using 4-8 technical replicates (repeated experiments run at the same time) from 2-4 biological replicates (using different virus stocks and different ACE2 variant preparations).

SARS-CoV-2 neutralization assay at biosafety level 3: Vero-E6 or Calu-3 cells were plated in a 96 well plate at 1.2×104 and 2.5×104 cells per well respectively and incubated overnight. At biosafety level 3, blocking or control (anti-GFP antibody) proteins and the Canadian clinical isolate of SARS-CoV-2 (V-2/Canada/VIDO-01/227 Feb. 2020) were mixed in fresh media supplemented with 3% fetal bovine serum (Gibco) and preincubated for 1 hr at 37° C. The cells were washed once with PBS and infected at the MOI of 0.1 with the proteins for 1 hr at 37° C. and 5% CO₂. Next, the mix was removed, and the cells were washed twice with PBS. Complete culture medium was added to each well, and cells were incubated at 37° C. and 5% CO2 for 24 hr followed by cell lysates collection for viral quantitation by qPCR. Mock cells were incubated with the culture supernatant from uninfected Vero-E6.

SARS-CoV-2 quantitative reverse-transcription PCR assay: For RNA analysis, total RNA was extracted using NucleoSpin RNA kit (Macherey-Nagel) following the manufacturer's protocol. Total RNA was reverse transcribed using 0.5-1 μg of total RNA and ImProm-II Reverse Transcriptase (Promega) according to the manufacturer's protocol. qRT-PCR was performed with PerfeCTa SYBR Green SuperMix (Quanta BioSciences) in the CFX96 Touch Real-Time PCR Detection System (Bio-Rad). The cycling conditions were 45 cycles of 94° C. for 30 sec, 55° C. for 60 sec, and 68° C. for 20 sec. Gene expression (fold change) was calculated using the 2(−ΔΔCT) method using human beta-actin mRNA transcript as the internal control. The following forward and reverse primer pairs were used for PCR: beta-actin 5′-TGGATCAGCAAGCAGGAGTATG-3′ (SEQ ID NO:34) and 5′-GCATTTGCGGTGGACGAT-3′ (SEQ ID NO:35), SARS-CoV-2 spike 5′-CAATGGTTTAACAGGCACAGG-3′ (SEQ ID NO:36) and 5′-CTCAAGTGTCTGTGGATCACG-3′ (SEQ ID NO:37) (see Zhou et al., 2020, “A pneumonia outbreak associated with a new coronavirus of probable bat origin,” Nature 579:270-273).

Cytotoxicity assay: The CellTiter-Glo Luminescent Cell Viability Assay (Promega) was used for quantitation of ATP in cultured cells. Cells lysates were assayed after mixing 100 μl of complete media with 100 μl of reconstituted CellTiter-Glo Reagent (buffer plus substrate) following the manufacturer's instructions. Samples were mixed by shaking the plates after which luminescence was recorded with a GloMax Explorer Model GM3510 (Promega) 10 min after adding the reagent.

### Example 2. Structural Modeling, Computational Protein Design, and Binding Affinity Testing

The soluble extracellular domain of hrACE2 (residues 18-614, ACE2(614)) was redesigned to bind the RBD of the SARS-CoV-2 spike protein using a combined computational/experimental protein engineering strategy. First, ACE2(614) was computationally redesigned using the Rosetta macromolecular modeling suite, and sets of mutations were introduced that improved the binding affinity of an ACE2(614)-Fc fusion protein for the SARS-CoV-2 spike RBD from 3- to 11-fold over the WT ACE2(614)-Fc protein in bio-layer interferometry (BLI) binding assays.

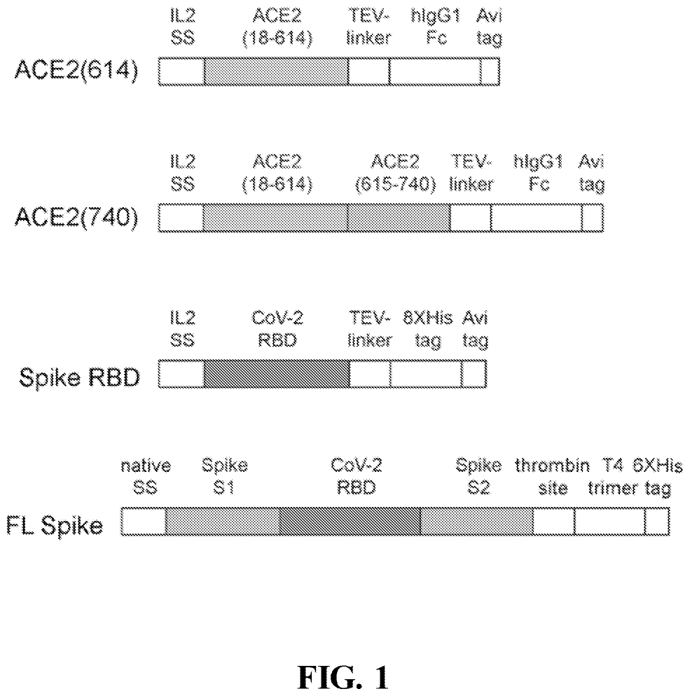

High-resolution ACE2-RBD structures (see Shang et al., 2020, “Structural basis of receptor recognition by SARS-CoV-2,” Nature 581:221-224 and Wang et al., 2020, “Structural and Functional Basis of SARS-CoV-2 Entry by Using Human ACE2,” Cell 181:894-904) show a large, flat binding interface primarily comprising the N-terminal helices of ACE2 (residues 18-90), with secondary interaction sites spanning residues 324-361 (data not shown; see FIG. 1A in Glasgow et al. 2020). To computationally redesign ACE2(614) for increased binding affinity with the RBD, the amino acid sidechains which are most crucial to the ACE2-RBD interaction (“hotspots”) were determined by performing a computational alanine scan on the binding interface using an established method in Rosetta (see Example 1). Then, a subset of hotspot residues in their local environment were systematically redesigned to generate models for new interfaces. ACE2 models were selected for testing that were most predicted to have improved binding to the RBD by their total energies and interface energies (data not shown; see FIGS. 1A-1C in Glasgow et al. 2020).

Computational alanine scanning suggested that the binding affinity of the ACE2-RBD interaction depends most crucially on 15 amino acids in the RBD contacting 18 amino acids in ACE2, 12 of which are in the most N-terminal ACE2 helix comprising residues 21 to 53 (data not shown). Of the ACE2 residues identified in the alanine scan, 6 amino acid sidechains (H34, Y41, Q42, Y83, K353, and D355) were determined to contribute most energetically to binding the spike RBD by the predicted change in binding energy upon mutation to alanine as assessed by DDG(complex) values greater than 1 Rosetta Energy Unit (REU) (see Example 1; see also FIG. 1A in Glasgow et al. 2020). To determine which of these “hotspots” to target for computational design, each hotspot residue was evaluated using two metrics: the per-residue energy and the contribution of the residue to the interface energy. Higher energies indicate lower stability. Hotspots H34, Q42 and K353 had per-residue energies in the top 30% of all ACE2 residues and were targeted for further design.

To determine whether point mutations at these positions could improve the ACE2-RBD binding affinity, computational saturation mutagenesis was performed at these positions (excluding mutations to cysteine to avoid disulfide bond formation) and the interface energy for each model was recalculated (data not shown; see FIG. 1B in Glasgow et al. 2020). Comparing the interface energy of each model to the wild-type complex interface energy revealed that no amino acid substitutions at positions 42 and 353 were predicted to be significantly stabilizing. However, several substitutions at position 34 were predicted to improve the interaction energy between ACE2 and the RBD (data not shown). Histidine 34 was mutated to a valine in the lowest-energy model because favorable hydrophobic interactions with leucine 455 in the RBD were predicted. In the highest-energy model, histidine 34 was mutated to an overly bulky isoleucine.

It was reasoned that both H34V and H34I, as the “best” and “worst” point mutants, were predicted to dramatically affect the interface energy in the context of their chemical environment, and that additional local mutations might improve binding affinity in both models to yield very different viable solutions. The Rosetta “Coupled Moves” flexible backbone design protocol was applied to redesign the local environment of V34 and 134 in each model (data not shown; see FIG. 1C in Glasgow et al 2020). ACE2 sidechains within 4 Å of residue 34 were allowed to mutate, while other ACE2 and RBD sidechains within 8 Å of ACE2 residue 34 could change rotamer and/or backbone conformations. This approach did not identify additional favorable mutations to H34V ACE2 in any of the models, but there were one to four additional mutations in the H341 ACE2 models. The lowest-energy designed protein based on H341 ACE2 had two additional mutations: K31F and E35Q (data not shown; see FIG. 1C in Glasgow et al. 2020). In this solution, ACE2 Q35 made a hydrogen bond with a repositioned RBD Q493, and ACE2 134 packed against a repositioned RBD L455. On the ACE2 side of the interface, F31 made a favorable hydrophobic interaction with the methylene in Q35. For both lowest-energy redesigned interfaces, the root-mean-square deviations (RMSD) of the mutable and repackable positions (atoms corresponding to ACE2 residues 29-39 and RBD residues 416-418, 452-456, and 492-494) in the model vs. the WT structure were less than 1 Å, and the total summed predicted pairwise interface energies for both design solutions were comparable (data not shown).

Next, the binding affinities of the computationally designed ACE2 variants were characterized as Fc fusions for spike RBD in BLI assays using purified proteins. Residues 18-614 of the extracellular domain of ACE2 (ACE2(614)) were transiently expressed with a C-terminal human IgG Fc domain fusion for improved affinity to spike (see Lei et al., 2020, “Neutralization of SARS-CoV-2 spike pseudotyped virus by recombinant ACE2-Ig,” Nat. Comm. 11:2070 and Li et al., 2020, “Potential host range of multiple SARS-like coronaviruses and an improved ACE2-Fc variant that is potent against both SARS-CoV-2 and SARS-CoV-1,” Microbiology preprint, doi:10.1101/2020.04.10.032342) in Expi293 cells (see Example 1). The BLI-measured binding affinities (K_{D}) of computationally-designed H34V ACE2(614)-Fc and K31F/H34I/E35Q ACE2(614)-Fc for the RBD were measured to be 3 and 11 times higher than the WT ACE2(614)-Fc, respectively (Table 1; data not shown but see FIG. 1E in Glasgow et al 2020). Binding of the RBD to H34V/N90Q/H374N/H378N ACE2(614)-Fc was also tested to determine the impact of removing a glycan that is adjacent to the interface at N90, as well as the protein's native peptidase activity. A recent deep mutational scanning (DMS) study reported enrichment for ACE2 variants with mutations at the N90 glycosylation site (see Procko et al., 2020, “Engineering human ACE2 to optimize binding to the spike protein of SARS coronavirus 2,” Science 369(6508):1261-1265, with preprints published on bioRxiv on Mar. 17, 2020, Apr. 6, 2020, and May 11, 2020, doi:10.1101/2020.03.16.994236); and histidines in position 374 and 378 together coordinate a Zn²⁺ ion necessary for enzymatic activity (data not shown). In the BLI assay, H34V/N90Q/H374N/H378N ACE2(614)-Fc demonstrated 10-fold improved affinity over WT ACE2(614)-Fc (Table 1; data not shown but see FIG. 1E in Glasgow et al. 2020).

TABLE 1
In vitro BLI measurements showing binding affinities of computationally
designed ACE2(614)-Fc receptor traps to RBD.

| ACE2(614)-Fc | | | |
|---|---|---|---|
| Construct | ACE2 variant | Replicate | K_{D} (nM) |
| CVD013 | Wild type | 1 | 10.0 ± 0.98 |
| | | 2 | 10.8 ± 1.9 |
| CVD014 | H34V | 1 | 4.3 ± 0.40 |
| | | 2 | 3.2 ± 0.36 |
| CVD019 | K31F/H34I/E35Q | 1 | 0.89 ± 0.11 |
| | | 2 | 1.03 ± 0.07 |
| CVD118 | H34V/N90Q/H374N/H378N | 1 | 0.95 ± 0.22 |
| | | 2 | 1.2 ± 0.20 |

* mutation inactivating the ACE2 peptidase function

### Example 3. Yeast Display Affinity Maturation of ACE2 Variants

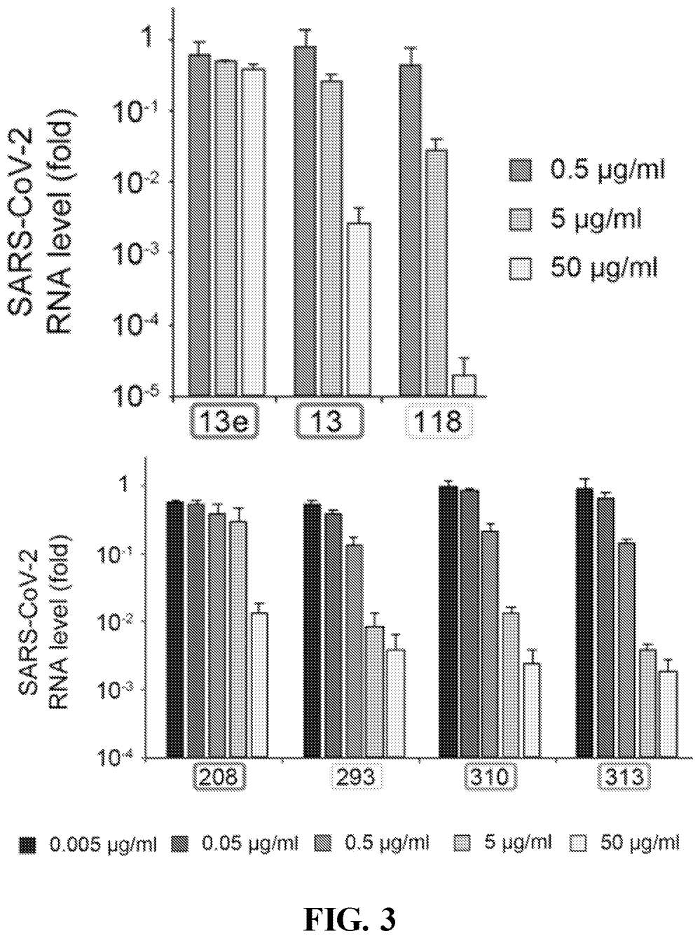

To further improve the binding affinity of the designed proteins for the spike RBD, a mutagenized library of ACE2(614) variants was expressed as Aga2p fusions for surface display in yeast cells, without the Fc domain to avoid avidity effects that might dominate affinity maturation. Four ACE2(614) variants were chosen as starting templates for a randomized yeast-displayed library, with the following mutations: H34V, N90Q, H34V/N90Q, and K31F/H34I/E35Q. Each of these variants, in addition to WT ACE2(614), were cloned as fusions to a myc tag, Aga2p, and C-terminal enhanced GFP for a simple readout of induction and surface display level (see FIG. 3A in Glasgow et al. 2020; see also Lim et al., 2017, “Dual display of proteins on the yeast cell surface simplifies quantification of binding interactions and enzymatic bioconjugation reactions,” Biotechnol. J. 12:1600696). The expression of WT ACE2(614) was first induced on EBY100 cells in SGCAA media at 20° C. and 30° C., and binding was confirmed using biotinylated spike RBD-Fc and streptavidin Alexa Fluor 647. Binding of the RBD was well-correlated with GFP expression, precluding the need for myc-tag (expression) staining (data not shown). Sixteen sublibraries of ACE2 residues 18-103 were made by homologous recombination into the ACE2(614) scaffold using the four input templates each mutagenized at four different rates using dNTP analogues (see Kariolis et al., 2014, “An engineered Axl “decoy receptor” effectively silences the Gash-Axl signaling axis,” Nat. Chem. Biol. 10:977-983). After transformation into EBY100 cells for a total library size of 2.7×10⁷ members, sequencing of 24 pre-sort clones showed an even distribution of mutations across the residues 18-103 with representation from all four input sequences (data not shown).

Sorts of increasing stringency were performed using different concentrations of RBD monomer as outlined in Table 2, below, and individual clones were analyzed along the way. Equilibrium sorts with decreasing RBD concentrations were used for sorts 1 to 3, and off-rate sorts with increasing dissociation times and decreasing gate size were used for the last two sorts. Sort 3 was performed with multiple binding stringencies and expression temperatures. For example, sorts of ACE2 induced at 30° C. did not show increased expression in subsequent rounds, but high affinity clones were observed from both 500 pM (for sort 3.1) and 200 pM (for sort 3.2) equilibrium sorts. The sequences of 21 clones from an 8 hour off-rate sort 4 did not show clonal convergence, but were enriched in favorable mutations in agreement with published DMS data (see Procko et al., 2020, “Engineering human ACE2 to optimize binding to the spike protein of SARS coronavirus 2,” Science 369(6508):1261-1265, with preprints published on bioRxiv on Mar. 17, 2020, Apr. 6, 2020, and May 11, 2020, doi:10.1101/2020.03.16.994236). A very stringent fifth sort, in which surface-displayed ACE2 were allowed to dissociate from RBD for 12 hours at room temperature and only 0.2% of the cell population were collected, still did not result in sequence convergence, but showed significant enrichment of one clone derived from the computationally designed K31F/H34I/E35Q ACE2(614) variant. The enriched ACE2(614) variant had the following seven mutations: Q18R, K31F, N33D, H34S, E35Q, W69R, and Q76R. Interestingly, a majority of sequences from sorts 4 and 5 were derived from the K31F/H34I/E35Q ACE2(614) parent, but many of these had mutations at 134 to serine or alanine (see Table 3 below). This suggested that while 134 led to an alternate design variant, the isoleucine was not always the ideal amino acid at this position. A small number of additional mutations appeared in variants originating from different parents, including F40L/S, N49D/S, M62T/I/V, and Q101R, while others appeared only in the K31F/H34I/E35Q background, such as L79P/F and L91P.

TABLE 2
Sort conditions for affinity maturation of ACE2 variants
by yeast surface display with increasing stringency.

| Sort | Sort type | Condition | Gate |
|---|---|---|---|
| 1 | Equilibrium sort | 50 nM RBD | 5% |
| 2 | | 5 nM RBD | 0.25% |
| 3.1 | | 0.5 nM RBD | 1% |
| 3.2 | | 0.2 nM RBD | 1% |
| 4 | Off-rate sort | 8 hr dissociation | 1% |
| 5 | | 12 hr dissociation | 0.2% |

Following sorts 3 through 5, 18-24 individual yeast clones were picked from each sort for further characterization. The sequences of the clones from sorts 4 and 5 are listed in Table 3, below. Deletions relative to the wild-type sequence are indicated using an underscored space. After growth and induction, each population was analyzed for high-affinity mutants by staining with decreasing concentrations of the RBD monomer. The best mutants from each sort were sequenced (mutations listed in Table 4) and their apparent K_{D} values for the monomeric spike RBD were measured by on-yeast titrations (Table 4; data not shown but see FIG. 3D in Glasgow et al. 2020). Apparent K_{D} values are reported as the average from the fit to all data in duplicate experiments with the errors of the fit (data not shown but see FIGS. 3D and 3E in Glasgow et al. 2020). Aga2p-GFP constructs were used in all yeast surface display experiments. These are listed alongside the ACE2(614)-Fc and ACE2(740)-Fc constructs that include the same affinity-enhancing mutations for convenience.

TABLE 3
Sequences from yeast display sorting rounds 4 and 5.

| SEQ ID NO | Sequence |
|---|---|
| SEQ ID NO: 41 | QSTIEEQARTFLDFFNIQAEDLSYQSSLASWNHNTNITEENVQNM |
| | NNAGDKWSAFLKKQSTLAQMYPLQEIQNLTVKLQLQALQQNGS |
| SEQ ID NO: 42 | QSTIEEQAKTFLDFFNIQAEDLFYQSSLASWNYNTDITEENVQNM |
| | NNAGDKWSAFLKEQSTLAQMYPLQEIRNLAVKLQLQALQQNGS |
| SEQ ID NO: 43 | QSTIEEQAKTFLDFFNIQAEDLFYQSSLASWNYNTNTTEKNVQNV |
| | NNAGDKWSAFLKEQGTLAQMYPLQEIQQPTVKLQLQALQQNGS |
| SEQ ID NO: 44 | QSTIEEQAKTFLDFFNIQAEDLFYQSSLASWNYNTNITEENVQNM |
| | NNAGDKWSAFLKEQSTLAQMYPLQEIQDPTVKLQLQALQQNGS |
| SEQ ID NO: 45 | QSTIEEQAKTFLDFFNIQAEDLFYQSSLASWNYNTNITEENVQNM |
| | NNAGDKWSAFLKEQSTLAQMYPLQEIQNPTVKLQLQALQQNGS |
| SEQ ID NO: 46 | QSTIEEQAKTFLDFFNIQAEDLFYQSSLASWNYNTNITEESVQNM |
| | NNAGDKWSAFLKEQSTLAQMYPLQEIQNPTVKLQLQALQQNGS |
| SEQ ID NO: 47 | QSTIEEQAKTFLDFFNIQAEDLFYQSSLASWNYNTNITEENVQNM |
| | NNAGDKWSALLKEQSTLAQMYPLQEIQNLTVKLQLQALQQNGS |
| SEQ ID NO: 48 | QSTIEEQAKTFLDFFNAQAEDLFYQSSLASWNYNTNITEENVQNM |
| | DNAGDKWSAFLKEQSTLAQMYPLQEIQNLTVKLQLQALQQNGS |
| SEQ ID NO: 49 | QSTIEEQAKTFLDFFNIRAEDLFYQSSLASWNYNTNITEENVQNM |
| | NNAGDRWSAFLKEQSTLAQMYPLQEIQNLTVKLQLQALQQNGS |
| SEQ ID NO: 50 | QSTVEEQAKTFLDFFNIRAEDLFYQSSLASWDYNTNITEENVQNM |
| | NNAGDKWSAFLKEQSTLAQMYPLQEIQNLTVKLQLQALQQNGS |
| SEQ ID NO: 51 | QSTIEEQAKTFLDKFNVEAEDLFYQSSLASWNYNTNVTEENVRN |
| | MNNAGDKWSAFLKEQSTLAQMYPLQEIQNLTVKLQLQALQQNG |
| | S |
| SEQ ID NO: 52 | QPTIEEQAKTFLDKFSVEAEDLLYQSSLASWDYNTNITEENVQNM |
| | NNAGDKWSAFLKEQSTLAQMYPLQEIQNLTVKLQLQAPQQNGS |
| SEQ ID NO: 53 | QSTIEEQVKAFLDKFNAEAEDLFYQSSLASWSYNTNITEANVQNM |
| | NNAGDKWSAFLKEQSTLAQMYPLQEIQNLTVKLQLQALQQNGS |
| SEQ ID NO: 54 | QSTIEEQVKAFLDKFNAEAEDLLYQSSLASWNCNTNITEENVQN |
| | MNNAGDKWSAFLKEQSTLAQMYPLQEIQNLTVKLQLQALQQNG |
| | S |
| SEQ ID NO: 55 | QSTIEEQAKTFLDKFNHEAEDLFYQSSLASWNYNTNITEENVQNM |
| | NNAGDKWSAFLREQSTLAQMYPLQEIQQLTVKLQLQALRQNGS |
| SEQ ID NO: 56 | QSTIEEQAKTFLDKFNAEAEDLSYRSSLASWDYNTNISEENVQNM |
| | NNAGDKWSAFLKEQSTLAQMYPLQEIQQLTVKLQLQALRQNGS |
| SEQ ID NO: 57 | QSTIEEQAKTFLDKFNVEAEDLFYQSSLASWNYNTNITEENVQNM |
| | NNAGDKWSAFLKEQSTLAQMYPLQEIQQLTVELQLQALQQNGS |
| SEQ ID NO: 58 | QSTIEEQAKTFLDFFNIQAEDLLYQSSLASWNYNTNITEENVQNM |
| | NNAGDKWSAFLKEQSTLAQMYPLQEIPNLTVKLRLQALQQNGS |
| SEQ ID NO: 59 | QSTIEEQAKTFLDFFNIQAEDLFYRSSLASWNYNTNITEENVQNM |
| | NNAGDKRSAFLKEQSTLAQMYPLQEIQQLTVKLQLQALRRNGS |
| SEQ ID NO: 60 | QSTIEEQAKAFLDKFNAEAEDLFYQSSLASWSYNTNITEENAQNM |
| | SNAGDRWSAFLKGQSTLAQMYPLQEIQQLTVKLQLQALQRNGS |
| SEQ ID NO: 61 | QPTIEEQARAFLDKFNAEAEDLFYQSGLASWNYNTNITEENVQNT |
| | NNAGDKWSAFLKEQSTLAQMYPLQEIQNLTVKLQLQALQQNGS |
| SEQ ID NO: 62 | PSTIEEQAKTFLDFFDIQAEDLFYQSSLVSWNYNTNITEENVQNM |
| | NNAGDTWSAFLKEQNTPAQMYPLQEIQNLTVKRQLQALQQNGS |
| SEQ ID NO: 63 | QSTIEEQAKTFLDFFDTQAEDLFYQSSLASWNYNTDITEENVQNM |
| | NNAGDKRSAFLKKQSTLAQMYPLQEIQNLTVKLQLQALQQNGS |
| SEQ ID NO: 64 | RSTIEEQAKTFLDFFDSQAEDLFYQSSLASWNYNTNITEENVQNM |
| | NNAGDKRSAFLKERSTLAQMYPLQEIQNLTVKLQLQALQQNGS |
| SEQ ID NO: 65 | QSTIEEQAKTFLDFFNIQAEDLFYQSSLASWNYNTNITEENVQNIN |
| | DAGGKWSAFLKEQSTLAQMYPLQEIQDLTVKLQLQALQQNGS |
| SEQ ID NO: 66 | QSTIEEQAKTFLDFFSIRAEDLFYQSSLASWNYNTNITEENVRNVN |
| | NAGDKWSAFLKEQSTLAQMYPLQEIQNLTVKLQLQALQQNGS |
| SEQ ID NO: 67 | QSTIEEQAKTFLDFFNIQAEDLLYQSSLASWNYNTNITEENVQNM |
| | NNAGDKWSAFLKEQSTLAQMYPLQEIQNLTVKLQLQAPQQNGS |
| SEQ ID NO: 68 | QSTIEEQAKTFLDFFNVQAEDLSYQSSMASWNYNTNITEENVQN |
| | MNNAGDKWSAFLKEQSTPAQMYPLQEIQNLTVKLQLQALQQNG |
| | S |
| SEQ ID NO: 69 | QSTIEEQAKTFLDFFNIQAEDLFYQSSLASWNYNTNITEENVQNM |
| | NNAGDKWSAFLKEQSTPAQMYPLQEIQNLTVKLQLQALQQNGS |
| SEQ ID NO: 70 | QSAIEEQAKTFLDFFNIQAEDLFYQSSLASWNYNTNITEENVQNM |
| | NNAGDKWSAFLKEQSTFAQMYPLQEIQNLTVKLQLQALQQNGS |
| SEQ ID NO: 71 | QSTIEEQAKTFLDFFDTQAEDLFYQSSLASWNYNTNITEENVQNM |
| | NNAGDKWSAFLKEQSTLAQMYPLQEIQNLTVKLQLQALQQNGS |
| SEQ ID NO: 72 | QSTIEEQAKTFLDFFSVQAEDLFYQSSLASWNYNTNITEENVQNM |
| | NNVGDKWSAFLKEQSTLAQMYPLQEIQNLTVKLQLQALQQNGS |
| SEQ ID NO: 73 | QSTIEEQAKTFLDFFSIQAEDLFYQSSLASWNYNTNITEENVQNM |
| | NNAGDKWSAFLKEQSTLAQMYPLQEIQNLTVKLQLQALQQNGS |
| SEQ ID NO: 74 | QSTIEEQAKTFLDFFNIQAEDLFYQSSLASWNYNTNITEENVQNM |
| | NNAGDKWSAFLKERSTLAQMYPLQEIQNLTVKLQLQALQQNGS |
| SEQ ID NO: 75 | QSTIEEQAKTFLDFFNIQAEDLFYQSSLASWNYNTNITEENVQNM |
| | NNAGDKWSAFLKEQSTLAQMYPLQEIQNLTVKLQLQALQQNGS |
| SEQ ID NO: 76 | QSTIEEQAKTFLDLSNLQAEDLFYQSGLASWNYNTNITGENVQNT |
| | DNAGDKWSAFLKEQSTLAQMYPLQEIQNLTVKLQL ALQQNGS |

The best-characterized mutants from sorts 3.1, 3.2, 4, and 5 had affinities of 0.52 nM, 0.45 nM, 0.19 nM, and 0.12 nM, respectively (between 39- and 170-fold higher affinity than WT ACE2(614)). Though each sort contained a variety of mutants, the highest-affinity clones contained N33D and H34S mutations and were derived from the K31F/H34I/E35Q ACE2(614) variant. The low likelihood of multiple base mutations in a single codon in error-prone PCR mutagenesis likely favored the I34S mutation from this background; interestingly, ACE2 from pangolin species that are hypothesized to be SARS-CoV-2 reservoirs also include a serine at position 34 (see Liu et al., 2020, “Composition and divergence of coronavirus spike proteins and host ACE2 receptors predict potential intermediate hosts of SARS-CoV-2,” J Med Virol 92:595-601). ACE2 N33 does not directly contact the RBD in the crystal structure, but the strong enrichment of the N33D mutation in the affinity maturation was consistent with the enrichment in the DMS study (see Procko et al., 2020, “Engineering human ACE2 to optimize binding to the spike protein of SARS coronavirus 2,” Science 369(6508):1261-1265, with preprints published on bioRxiv on Mar. 17, 2020, Apr. 6, 2020, and May 11, 2020, doi:10.1101/2020.03.16.994236).

TABLE 4
Apparent binding affinities of ACE2(614) variants measured on
the surface of yeast with monomeric SARS-CoV-2 spike RBD.

| | | Apparent K_{D} | Aga2p-GFP | ACE2(614)- | ACE2(740)- |
|---|---|---|---|---|---|
| Mutations | Origin | (nM) | construct | Fc construct | Fc construct |
| — | WT | 20.4 ± 1.8 | Y208 | CVD013 | CVD208 |
| H34V | Computational | 9.29 ± 0.78 | Y295 | CVD014, | CVD295 |
| | design | | | CVD127* | |
| N90Q | Procko et al. | 4.54 ± 0.55 | Y117 | CVD117 | |
| K31F, H34I, | Computational | 1.71 ± 0.02 | Y293 | CVD019 | CVD293 |
| E35Q | design | | | | |
| H34V, N90Q | Computational | 5.47 ± 0.68 | Y292 | CVD118*, | CVD292 |
| | design | | | CVD278† | |
| A25V, T27Y, | DMS-guided | 0.40 ± 0.03 | Y310 | | CVD310† |
| H34A, F40D | design | | | | |
| K31Y, W69V, | DMS-guided | 0.64 ± 0.05 | Y311 | | CVD311† |
| L79T, L91P | design | | | | |
| T27Y, H34A, | DMS-guided | 0.84 ± 0.09 | Y312 | | CVD312† |
| N90Q | design | | | | |
| S19P, Q42L, | DMS-guided | 0.94 ± 0.09 | Y355 | | CVD355† |
| L79T, N90Q | design | | | | |
| K31F, N33D, | Yeast display, | 0.52 ± 0.04 | Y313 | | CVD313† |
| H34S, E35Q | round 3.1 | | | | |
| K31F, N33D, | Yeast display, | 0.45 ± 0.18 | Y354 | | CVD354† |
| H34A, E35Q, | round 3.2 | | | | |
| N49D, N51S, | | | | | |
| N53S, E57G, | | | | | |
| N64D | | | | | |
| T27A, K31F, | Yeast display, | 0.19 ± 0.02 | Y353 | | CVD353† |
| N33D, H34S, | round 4 | | | | |
| E35Q, N61D, | | | | | |
| K68R, L79P | | | | | |
| S19P, N33S, | Yeast display, | 0.61 ±0.04 | Y375 | | CVD375† |
| H34V, F40L, | round 4 | | | | |
| N49D, L100P | | | | | |
| Q18R, K31F, | Yeast display, | 0.12 ±0.01 | Y373 | | CVD373† |
| N33D, H34S, | round 5 | | | | |
| E35Q, W69R, | | | | | |
| Q76R | | | | | |

*includes H374N/H378N inactivation mutations
†includes H345L inactivation mutation

### Example 4. DMS-Guided Computational Design

As an orthogonal approach to generate affinity-enhanced ACE2 variants, the results from the DMS experiment by Procko were leveraged to perform an additional round of DMS-guided computational design. The computational design strategy described in Example 2 targeted alanine scan hotspots. Procko's DMS experiment identified beneficial ACE2 point mutations in the ACE2-RBD interface at non-hotspot positions, which could improve binding affinity by direct interactions with the RBD, as well as outside the binding interface, which might serve a stabilizing role. These two classes of mutations would not have been predicted by the computational design strategy described in Example 2. Thus, another round of computational saturation mutagenesis was performed at non-hotspot ACE2 positions in the ACE2-RBD interface (A25, T27, K31) as well as the non-interface residue W69, to predict additional mutations. A set of DMS-guided, computationally designed ACE2 variants were generated with 3-4 mutations each that included at least two mutations outside the interface chosen directly from the DMS dataset, combined with 1-2 mutations from the computational saturation mutagenesis at non-hotspot positions that were also enriched in DMS (Table 4). These designed proteins were displayed on the surface of yeast as ACE2(614)-Aga2p fusions. The K_{D,app} of the DMS-guided ACE2(614) variants for the monomeric RBD was measured to be between 0.4 and 1 nM by on-yeast titrations, which is between 21- and 51-fold higher than WT ACE2(614) (Table 4; data not shown but see FIG. 3E in Glasgow et al. 2020). The ACE2(614) variant from DMS-guided computational design measured to have the lowest apparent K_{D} included the following mutations: A25V, T27A, H34A, and F40D.

### Example 5. Addition of Collectrin Domain and Stability Improvement

BLI was used to test whether inclusion of the natural C-terminal ACE2 collectrin domain (residues 614-740) could improve the protein's binding affinity for spike. General expression constructs for ACE2 with and without the collectrin domain, as well as expression constructs for spike RBD and full-length spike are shown in FIG. 1. A recent cryo-EM structure shows ACE2 as a dimer, with the collectrin domain connecting the extracellular peptidase domain of ACE2 to its transmembrane helix (see Yan et al., 2020. “Structural basis for the recognition of SARS-CoV-2 by full-length human ACE2,” Science 367:1444-1448; data not shown but see also Supplemental Figure S7 in Glasgow et al. 2020). The structure also reveals additional inter-collectrin domain contacts and C-terminal contacts between peptidase domains. Fc fusions of WT ACE2 containing the collectrin domain were also recently shown to be more effective in blocking viral infection (see Li et al., 2020, “Potential host range of multiple SARS-like coronaviruses and an improved ACE2-Fc variant that is potent against both SARS-CoV-2 and SARS-CoV-1,” Microbiology preprint, doi:10.1101/2020.04.10.032342), perhaps by repositioning the ACE2 monomers for improved binding to spike. Furthermore, spike is a homotrimer containing three RBDs, each of which can independently bind ACE2 (see Wrapp et al., 2020, “Cryo-EM structure of the 2019-nCoV spike in the prefusion conformation,” Science 367:1260-1263 and Zhou et al., 2020, “A pH-dependent switch mediates conformational masking of SARS-CoV-2 spike,” Immunology, doi:10.1101/2020.07.04.187989). It was hypothesized that the inclusion of the ACE2 collectrin domain and the additional two spike RBDs would increase the strength of the ACE2-spike interaction through stabilization and avidity effects. Binding of ACE2-Fc with the collectrin domain (ACE2(740)-Fc) to spike RBD and full-length spike (FL spike) was tested using BLI.

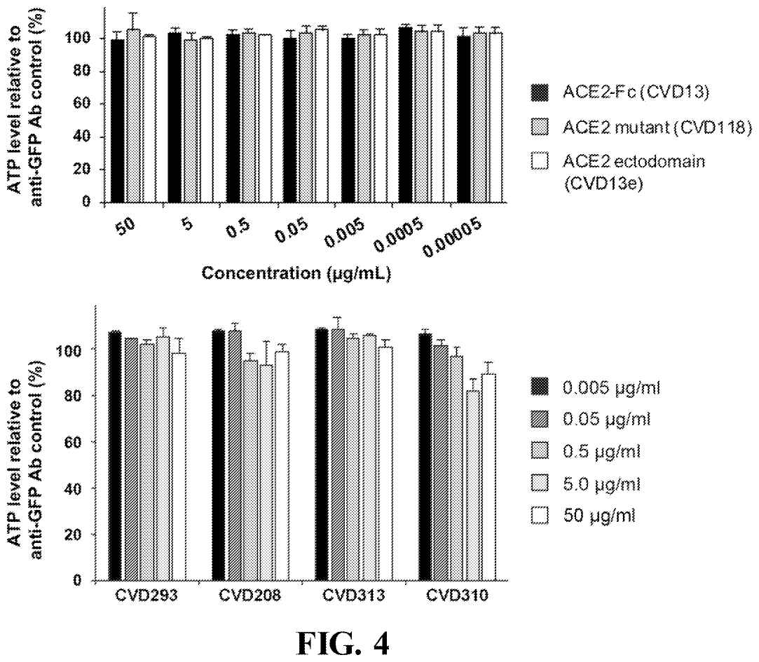

Compared to the ACE2(614)-Fc interaction with the spike RBD, the monovalent K_{D} of WT ACE2(740)-Fc for the spike RBD was decreased 3.7-fold, and the K_{D} of ACE2-Fc with or without the collectrin domain for FL spike decreased dramatically (Table 5; data not shown but see FIG. 4B and Supplemental Figures S8A-S8E in Glasgow et al. 2020). Both WT and variant ACE2(740)-Fc binding interactions with the FL spike were too tight to be accurately measured by BLI due to the substantially decreased off-rates (Table 5). ACE2(740)-Fc variants from DMS-guided design and affinity maturation in yeast had greatly reduced off-rates for monovalent interactions with the RBD (Table 5; data not shown but see Supplemental Figures S8F and S8G in Glasgow et al. 2020). The highest-affinity mutants from the yeast display campaign described in Example 3 were poorly expressed as Fc fusions, indicating that despite numerous reports of stabilizing mutations from yeast display, ACE2 expression on yeast did not translate to well-folded soluble protein.

TABLE 5
Binding of ACE2-Fc variants with and without collectrin
domain to spike RBD and full-length spike.

| | | | Spike RBD | Full-length spike |
|---|---|---|---|---|
| ID | Mutations | Scaffold | binding (K_{D}) | binding (K_{D}) |
| CVD013 | — | ACE2(614)-Fc | 10.8 nM | Not measurable |
| | | | | (too tight) |
| CVD208 | — | ACE2(740)-Fc | 2.8 nM | Not measurable |
| | | | | (too tight) |
| CVD014 | H34V | ACE2(614)-Fc | 4.3 nM | Not measurable |
| | | | | (too tight) |
| CVD019 | K31F, H34I, | ACE2(614)-Fc | 0.89 nM | Not measurable |
| | E35Q | | | (too tight) |
| CVD118 | H34V, N90Q, | ACE2(614)-Fc | 0.95 nM | Not measurable |
| | H374N*, H378N* | | | (too tight) |
| CVD292 | H34V, N90Q | ACE2(740)-Fc | 0.45 nM | Not measurable |
| | | | | (too tight) |
| CVD293 | K31F, H34I, | ACE2(740)-Fc | 0.23 nM | Not measurable |
| | E35Q | | | (too tight) |
| CVD310 | A25V, T27Y, | ACE2(740)-Fc | Not measurable | Not measured |
| | H34A, F40D, | | (too tight) | |
| | H345L* | | | |
| CVD313 | K31F, N33D, | ACE2(740)-Fc | Not measurable | Not measured |
| | H34S, E35Q, | | (too tight) | |
| | H345L* | | | |

*mutation inactivating the ACE2 peptidase function

The soluble domain of ACE2 converts angiotensin II to angiotensin(1-7), a vasodilator, and was shown to be safe in clinical trials (see Haschke, et al., 2013, “Pharmacokinetics and pharmacodynamics of recombinant human angiotensin-converting enzyme 2 in healthy human subjects,” Clin. Pharmacokinet. 52:783-792 and Khan et al., 2017, “A pilot clinical trial of recombinant human angiotensin-converting enzyme 2 in acute respiratory distress syndrome,” Crit. Care Lond. Engl. 21:234). The RBD binds outside the ACE2 enzyme active site. The H374N/H378N mutations were introduced to inactivate the peptidase activity of ACE2 to avoid off-target vasodilation effects by ablating Zn2+ binding without affecting the binding affinity for the RBD (see Lei et al., 2020, “Neutralization of SARS-CoV-2 spike pseudotyped virus by recombinant ACE2-Ig,” Nat Comm 11:2070). However, protein stability is also an important factor to consider in engineering an optimal ACE2-based therapeutic scaffold. Incorporation of the ACE2 collectrin domain in the Fc-fused constructs improved the apparent melting temperature of the ACE2-Fc variants as measured by circular dichroism spectroscopy, but the H374N/H378N enzymatic inactivation mutations were significantly destabilizing (Table 6; data not shown but see Supplemental Figure S9 in Glasgow et al. 2020). Therefore, the ACE2(740)-Fc scaffold was adapted to include the inactivation mutation H345L instead, which is important for substrate binding and is not destabilizing (Table 6; data not shown but see Supplemental Figure S9 in Glasgow et al. 2020; see also Guy et al., 2005, “Identification of critical active-site residues in angiotensin-converting enzyme-2 (ACE2) by site-directed mutagenesis,” FEBS J. 272:3512-3520). H345L ACE2(740)-Fc does not have detectable peptidase activity in an activity assay and does not impact binding to the spike RBD in a BLI assay (data not shown; see Supplemental Figure S10 in Glasgow et al. 2020), but maintains the thermal stability of WT ACE2.

TABLE 6
Thermostability of ACE2-Fc constructs measured
by circular dichroism spectroscopy.

| | | | Apparent melting |
|---|---|---|---|
| Construct | Mutations | Scaffold | temperature (° C.) |
| CVD118 | H34V, N90Q, | ACE2(614)-Fc | 47.3 |
| | H374N*, H378N* | | |
| CVD127 | H34V, H374N*, | ACE2(614)-Fc | 48.9 |
| | H378N* | | |
| CVD013 | — | ACE2(614)-Fc | 51.8 |
| CVD278 | H34V, N90Q, | ACE2(614)-Fc | 52.7 |
| | H345L* | | |
| CVD013e | — | ACE2(614) | 53.1 |
| | | ectodomain | |
| CVD019 | K31F, H34I, | ACE2(614)-Fc | 54.1 |
| | E35Q | | |
| CVD208 | — | ACE2(740)-Fc | 55.3 |
| CVD293 | K31F, H34I, | ACE2(740)-Fc | 50.0 |
| | E35Q | | |
| CVD313 | K31F, N33D, | ACE2(740)-Fc | 50.2 |
| | H34S, E35Q, | | |
| | H345L* | | |
| CVD294 | K31F, H34I, | ACE2(740)-Fc | 52.5 |
| | E35Q, N90Q | | |
| CVD292 | H34V, N90Q | ACE2(740)-Fc | 52.6 |

*mutation inactivating the ACE2 peptidase function

### Example 6. Characterization of variants using neutralization assays

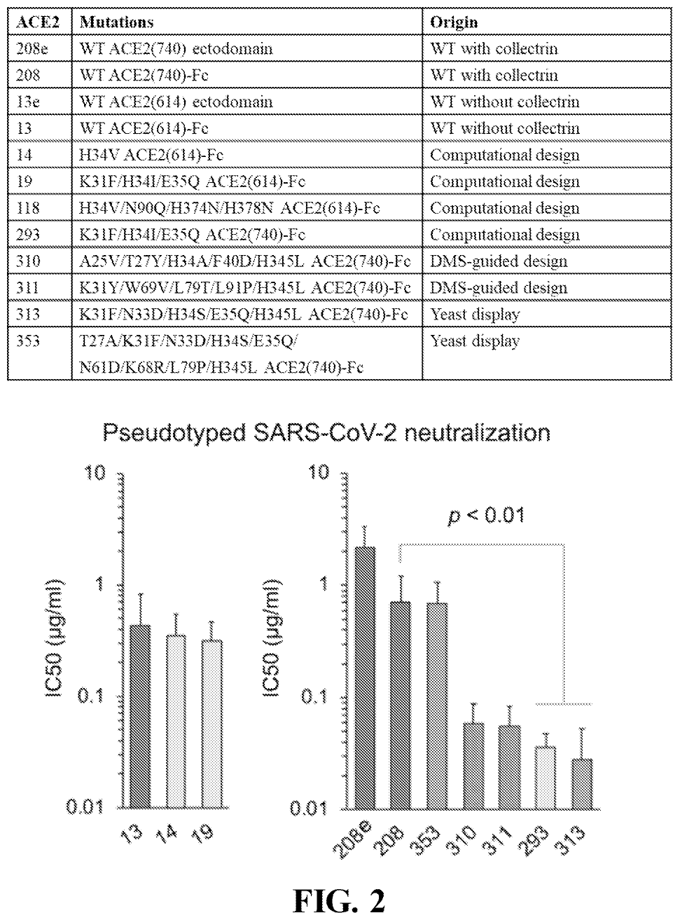

The binding affinity improvements to ACE2 (see Examples 2-5) were robust to the method of measurement (BLI vs. binding on yeast) and well-correlated with neutralization efficacy. To evaluate their efficacy in neutralizing SARS-CoV-2 infections, several affinity-improved ACE2 variants from DMS-guided design and yeast display were expressed in the H345L ACE2(740)-Fc format, purified, and assayed for viral neutralization against pseudotyped lentivirus and authentic SARS-CoV-2 (FIGS. 2 and 3). In the pseudotyped viral neutralization assay in ACE2-expressing HEK cells, different ACE2(740)-Fc molecules with mutations derived from computational design, DMS-guided design, and affinity maturation using yeast surface display neutralized SARS-CoV-2 with IC50 values of 58 ng/mL, 55 ng/mL, 36 ng/mL, and 28 ng/mL (Table 7, FIG. 2, bottom panel, see variants 310/311, 293, and 313, respectively), demonstrating multiple paths to significant improvements in efficacy. It was also confirmed that the misfolded affinity-enhanced ACE2 variant 353 (T27A/K31F/N33D/H34S/E35Q/N61D/K68R/L79P/H345L ACE2(740)-Fc) does not effectively neutralize SARS-CoV-2, despite this molecule's enrichment in the yeast display campaign and low K_{D,app}, possibly because it is unstable or otherwise misfolded (FIG. 2, bottom panel; data not shown but see Supplemental Figure S11 in Glasgow et al. 2020). Taken together, the neutralization data revealed that the mutations to ACE2 from computational design and affinity maturation, addition of the collectrin domain, and fusion to the Fc domain together significantly improve neutralization over unmodified ACE2(740).

TABLE 7
IC50 of ACE2 variants measured in neutralization assays.

| | | | IC50 | IC50 |
|---|---|---|---|---|
| | | | (μg/ml), | (μg/ml), |
| ID | Mutations | Scaffold | pseudovirus | VeroE6 |
| CVD013 | — | ACE2(614)-Fc | 0.43 ± 0.39 | |
| CVD208 | — | ACE2(740)-Fc | 0.71 ± 0.51 | |
| CVD208e | — | ACE2(740) | 2.19 ± 1.15 | |
| | | ectodomain | | |
| CVD014 | H34V | ACE2(614)-Fc | 0.35 ± 0.19 | |
| CVD019 | K31F, H34I, E35Q | ACE2(614)-Fc | 0.31 ± 0.16 | |
| CVD118 | H34V, N90Q, | ACE2(614)-Fc | | <0.5 |
| | H374N*, H378N* | | | |
| CVD293 | K31F, H34I, E35Q | ACE2(740)-Fc | 0.036 ± 0.01 | 0.136 ± 0.08 |
| CVD310 | A25V, T27Y, H34A, | ACE2(740)-Fc | 0.058 ± 0.03 | 0.089 ± 0.01 |
| | F40D, H345L* | | | |
| CVD311 | K31Y, W69V, L79T, | ACE2(740)-Fc | 0.055 ± 0.03 | |
| | L91P, H345L* | | | |
| CVD313 | K31F, N33D, H34S, | ACE2(740)-Fc | 0.028 ± 0.02 | 0.073 ± 0.02 |
| | E35Q, H345L* | | | |
| CVD353 | T27A, K31F, N33D, | ACE2(740)-Fc | 0.69 ± 0.38 | |
| | H34S, E35Q, N61D, | | | |
| | K68R, L79P, H345L* | | | |

*mutation inactivating the ACE2 peptidase function

Authentic SARS-CoV-2 neutralization assays in VeroE6 cells in a biosafety level 3 facility closely reflected the results from the pseudotyped viral neutralization assays. Fusion of the Fc domain to ACE2(614) improved neutralization by 370-fold over monomeric ACE2(614), and additional inclusion of computationally-predicted mutations H34V and N90Q improved neutralization by more than 50,000-fold over monomeric ACE2(614), at the highest concentration tested (FIG. 3, top panel, 50 μg/ml). The IC50 for this affinity-enhanced ACE2(614)-Fc variant, known as CVD118, was less than 0.5 μg/ml.

Addition of the ACE2 collectrin domain further improved neutralization potency, with ACE2(740)-Fc variants originating from computational design, DMS-guided design, and affinity maturation in yeast demonstrating efficient neutralization in the neutralization assay using bona fide SARS-CoV-2 (FIG. 3, bottom panel). Variants 118 and 292 have the same computationally-designed affinity-enhancing mutations (H34V and N90Q), but variant 292 also includes the collectrin domain, and neutralizes SARS-CoV-2 with 13-fold improved potency over variant 118 at the highest concentration tested (data not shown; see FIG. 3F, 10 μg/mL in U.S. Provisional Application No. 63/058,379). Variant 293 also includes the collectrin domain and the computationally-predicted mutations K31F, H34I, and E35Q, and has 774-fold improved potency over variant 118 at the highest concentration tested (data not shown; see FIG. 3F, 10 μg/mL in U.S. Provisional Application No. 63/058,379). The IC50 for ACE2(740)-Fc variant 293 was less than 0.1 μg/ml (data not shown). WT ACE2(740)-Fc (variant 208), computationally designed variant 293, DMS-guided design 310, and yeast affinity-matured variant 313 were tested at concentrations from 0.005 to 50 μg/mL. Variants 293, 310, and 313 each considerably diminished viral RNA levels at concentrations starting at 0.05 μg/mL, while WT ACE2(740)-Fc only had neutralization efficacy at 5 μg/mL. Variants 310 and 313 displayed the most neutralization potency, with IC50s of approximately 89 ng/mL (e.g., approximately 90 ng/mL) and approximately 73 ng/mL, respectively (FIG. 3, bottom panel and Table 7). This neutralization potency is comparable with recently reported antibodies isolated from convalescent COVID-19 patients (see Robbiani et al., 2020, “Convergent antibody responses to SARS-CoV-2 in convalescent individuals,” Nature, 1-8 and Brouwer et al., 2020, “Potent neutralizing antibodies from COVID-19 patients define multiple targets of vulnerability,” Science, doi:10.1126/science.abc5902). None of the ACE2 variants induced cytotoxicity in uninfected cells at the concentrations used in the neutralization assay (FIG. 4). Additional live SARS-CoV-2 neutralization experiments with shorter incubation times (16 h rather than 26 h) and a different SARS-CoV-2 strain were conducted in a different laboratory to ensure reproducibility and measure the effect of the affinity-enhanced ACE2(740)-Fc molecules on viral entry more directly and yielded similar results: IC50 values were in the range of 0.1 to 1 μg/mL for variant 293 and lower for variants 310 and 313 (data not shown; see Supplemental Figure S13 in Glasgow et al. 2020). Live virus qPCR assays of three viral genes (N, E, RdRp) were run 16 hours post-infection using host genes BGUS or ACTB as normalization controls. K31F/H34I/E35Q ACE2(740)-Fc (variant 293) had an IC50 between 0.1 and 1 μg/ml. A25V/T27Y/H34A/F40D/H345L ACE2(740)-Fc (variant 310) and K31F/N33D/H34S/E35Q/H345L ACE2(740)-Fc (variant 313) neutralized completely at 1 μg/ml.

The inclusion of the ACE2 collectrin domain and the human IgG Fc domain dramatically increased the neutralization potency of the ACE2 variants through improved affinity, stability, and avidity. The Fc fusion results in ACE2 dimerization, but the collectrin domain may serve to position the ACE2 molecules closer together than would be achieved with the Fc alone (see Yan et al., 2020, “Structural basis for the recognition of SARS-CoV2 by full-length human ACE2,” Science 367:1260-1263). The interaction of dimeric ACE2-Fc with the full-length trimeric spike protein is also stronger than with the monomeric RBD due to avidity effects. As a combined result of these effects, the use of the H345L ACE2(740)-Fc scaffold is central to the neutralization potency of the affinity-enhanced variants.

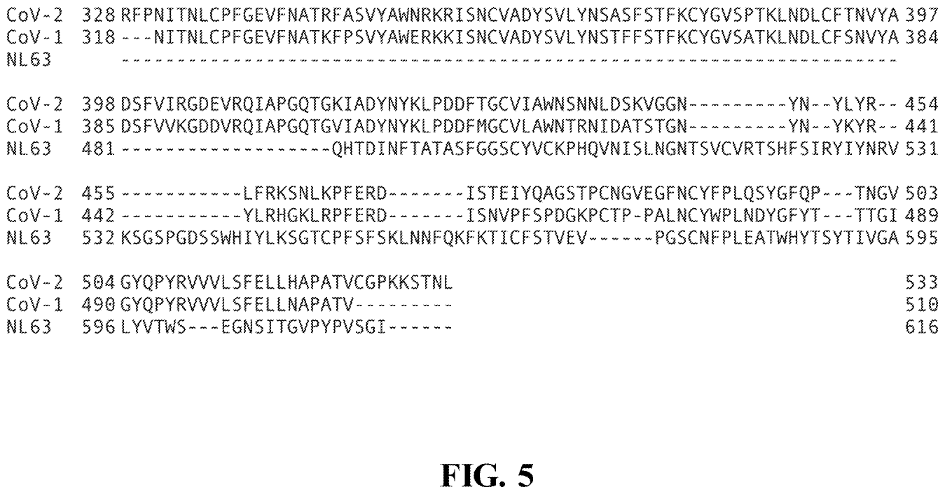

ACE2-based therapeutics could be used to treat other respiratory infections with ACE2-dependent cell entry mechanisms, such as those caused by SARS-CoV-1 and HCoV-NL63 (i.e. NL63) coronaviruses (Zhou et al., 2020, “A pneumonia outbreak associated with a new coronavirus of probable bat origin,” Nature 579:270-273; Wang et al., 2013, “Structure of MERS-CoV spike receptor-binding domain complexed with human receptor DPP4,” Cell Res 23:986-993). Thus, WT ACE2(740)-Fc and the most robustly expressed ACE2(740)-Fc variants were tested for binding to the SARS-CoV-1 and NL63 spike RBD domains. Indeed, wild-type ACE2(740)-Fc (variant 208), a receptor trap from affinity maturation in yeast (variant 313, with K31F, N33D, H34S, E35Q, and enzymatic inactivation mutation H345L), and its computationally designed parent (variant 293, K31F, H34I, E35Q) bound with nanomolar K_{D} to the SARS-CoV-1 RBD and tens of nanomolar K_{D} to the NL63 RBD (Table 8; data not shown but see FIG. 5 in Glasgow et al. 2020), which is close to previous observations for the NL63 RBD-WT ACE2 interaction (Wu et al., 2009, “Crystal structure of NL63 respiratory coronavirus receptor-binding domain complexed with its human receptor,” Proc Natl Acad Sci 106:19970-19974). The lower binding affinity for the NL63 RBD interaction with ACE2-Fc is likely due to its relatively low structure/sequence similarity to the SARS-CoV-2 RBD; by contrast, the SARS-CoV-1 and SARS-CoV-2 RBDs are structurally homologous and have 73% sequence identity (FIG. 5; data not shown but see Supplemental Figure S14 in Glasgow et al. 2020). In contrast, these molecules did not bind appreciably to the Middle East respiratory syndrome (MERS) RBD up to 150 nM (data not shown; see Supplemental Figure S15 in Glasgow et al. 2020), which enters cells through interactions with the dipeptidyl pepdidase IV (DPP4, also known as CD26) membrane protein.

TABLE 8
Binding of ACE2-Fc variants to spike RBD from SARS-CoV-2, SARS-CoV-1, and NL63.

| | | | SARS-CoV-2 | SARS-CoV-1 | NL63 |
|---|---|---|---|---|---|
| | | | RBD binding | RBD binding | RBD binding |
| ID | Mutations | Scaffold | (K_{D}) | (K_{D}) | (K_{D}) |
| CVD208 | — | ACE2(740)-Fc | 3.0 nM | 15 nM | 22 nM |
| CVD293 | K31F, H34I, E35Q | ACE2(740)-Fc | 0.23 nM | 5.3 nM | 11 nM |
| CVD313 | K31F, N33D, | ACE2(740)-Fc | Not measurable | 3.9 nM | 37 nM |
| | H34S, E35Q, H345L* | | (too tight) | | |

*mutation inactivating the ACE2 peptidase function

### Example 7. Characterization of ACE2 Mutant Enzymatic Activity

Example 7 is based on experiments described in Glasgow et al., 2021, Reply to Liu et al., “Specific mutations matter in specificity and catalysis in ACE2,” letter in Proc. Nat. Acad. Sci. 118(15):e2024450118, doi:10.1073/pnas.2024450118, which was published in response to Liu et al., 2021, “His345 mutant of angiotensin-converting enzyme 2 (ACE2) remains enzymatically active against angiotensin II,” letter in Proc. Nat. Acad. Sci. 118(15):e2023648118, doi:10.1073/pnas.2023648118. The Liu et al. 2021 letter is a response to Glasgow et al. 2020.

Understanding the physiologically relevant ACE2 peptidase activity determinants is useful, as several groups are developing ACE2-based receptor traps with intact, modestly attenuated, or ablated activity to separate the effects of blocking on angiotensin II (Ang II) conversion. (see Monteil et al., 2020, “Inhibition of SARS-CoV-2 infections in engineered human tissues using clinical-grade soluble human ACE2,” Cell 181:905-913; Lei et al., 2020, “Neutralization of SARS-CoV-2 spike pseudotyped virus by recombinant ACE2-Ig,” Nat. Commun. 11:2070; Chan et al., 2020, “Engineering human ACE2 to optimize binding to the spike protein of SARS coronavirus 2,” Science 369:1261-1265; Glasgow et al., 2020, “Engineered ACE2 receptor traps potently neutralize SARS-CoV-2,” Proc. Nat. Acad. Sci. 117:28046-28055; Iwanaga et al., 2020, “Novel ACE2-IgG1 fusions with improved activity against SARS-CoV2,” bioRxiv, doi:10.1101/2020.06.15.152157; and Tada et al., 2020, “A soluble ACE2 microbody protein fused to a single immunoglobulin Fc domain is a potent inhibitor of SARS-CoV-2 infection in cell culture,” bioRxiv, doi:10.1101/2020.09.16.300319).

Toward this end, ACE2 variants containing an H345L mutation were generated. This mutation is postulated to form part of the oxyanion binding site (see Guy et al., 2005, “Identification of critical active-site residues in angiotensin-converting enzyme-2 (ACE2) by site-directed mutagenesis,” FEBS J. 272:3512-3520). Mutations in the oxyanion hole for zinc metalloproteases are well known to disrupt the tetrahedral oxyanion in the transition state and dramatically reduce activity (see Cerdà-Costa et al., 2014, “Architecture and function of metallopeptidase catalytic domains,” Protein Sci. 23:123-144). Indeed, the H345L mutation was reported, herein and by others, to reduce activity in ACE2 over 300-fold against a commonly used fluorogenic peptide mimic (Dnp-APK(Mca)) as a proxy for ACE2 activity (Guy et al., 2005, “Identification of critical active-site residues in angiotensin-converting enzyme-2 (ACE2) by site-directed mutagenesis,” FEBS J. 272:3512-3520). Another mutant at this same site, H345A, was similarly inactive on Dnp-APK(Mca) but retained or even had enhanced activity on Ang II (see Liu et al., 2021, “His345 mutant of angiotensin-converting enzyme 2 (ACE2) remains enzymatically active against angiotensin II,” Proc. Nat. Acad. Sci., doi:10.1073/pnas.2023648118).

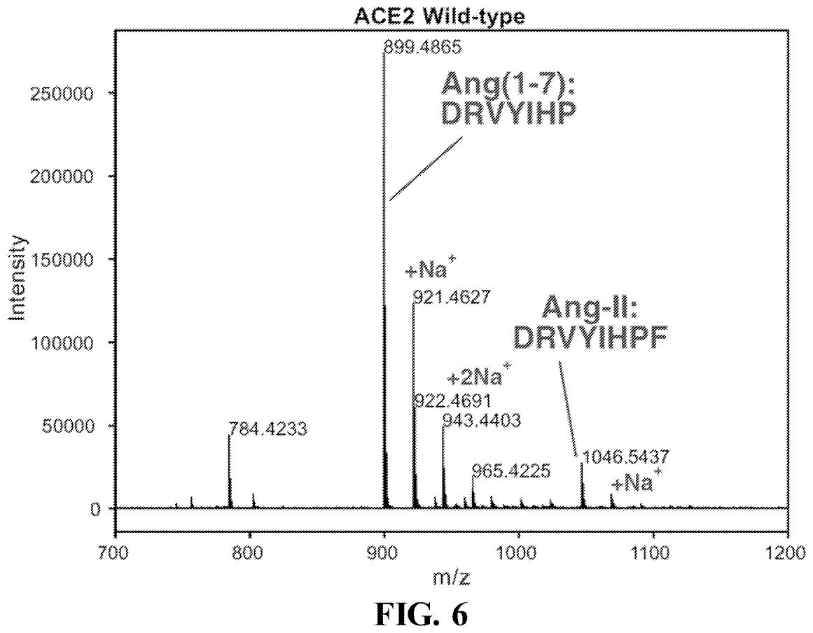

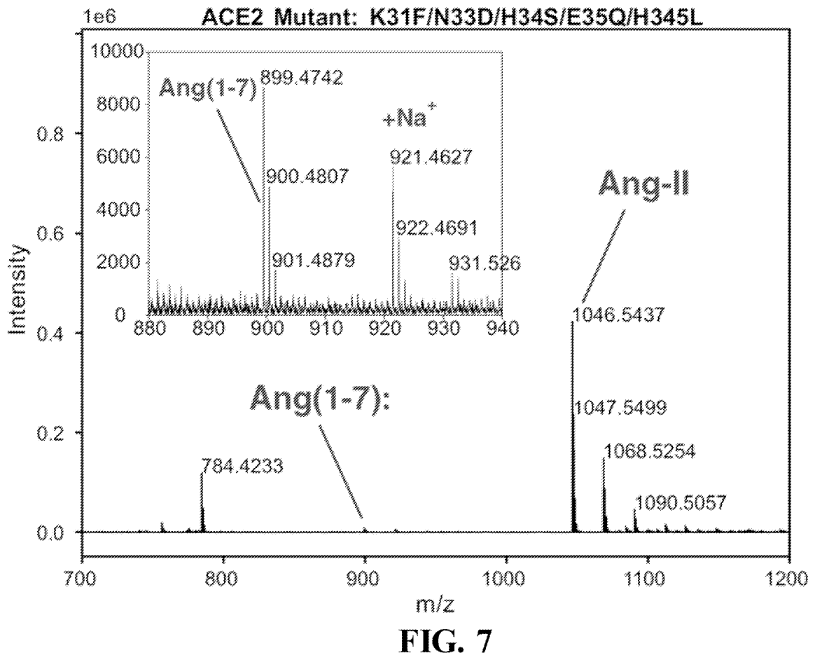

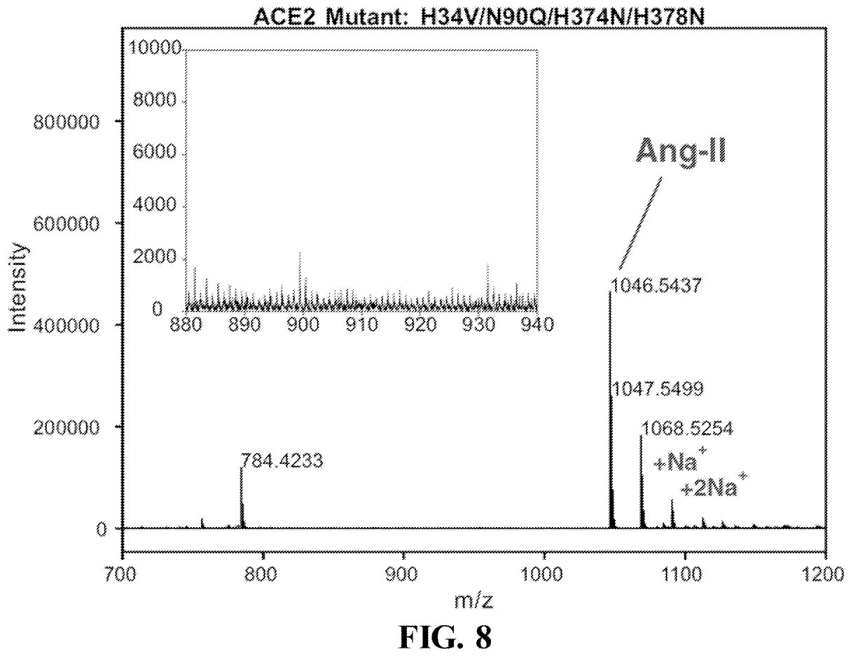

To confirm that the ACE2 H345L mutation does not retain peptidase activity, the lead ACE2 receptor trap, CVD313 (K31F/N33D/H34S/E35Q/H345L), was tested for ability to hydrolyze Ang II alongside controls including CVD208 (wild-type ACE2(740)-Fc) and a Zn2+-binding ablated mutant CVD118 (ACE2(614)-Fc-H34V/N90Q/H374N/H378N). It was found that CVD313 has very little activity against Ang II (FIGS. 6, 7, and 8). For example, under conditions where the wild-type CVD208 cleaved >90% of Ang II (FIG. 6) to yield the cleavage product Ang(1-7), for CVD313, vanishingly small (<1%) levels of Ang(1-7) were observed (FIG. 7). No formation of any Ang(1-7) using CVD118 was observed, confirming the necessity of Zn2+ in the active site (FIG. 8).

There are various possible reason that could account for the differences in substrate-dependent activity of ACE2 H345L (inactive on both substrates) versus ACE2 H345A (inactive on Dnp-APK(Mca) but active on Ang II). It is possible the differences depend on the other mutations in CVD313. Alternatively, Ang II contains a P2 His residue not found in Dnp-APK(Mca), which could possibly cooperate with H345 to form part of the oxyanion binding (data not shown; see FIG. 1D in Glasgow et al. 2021 letter). Some years ago, substrate-assisted catalysis was observed where mutation of the catalytic H64A in subtilisin reduced activity by 106 against standard substrates, but was substantially restored by substrates containing a P2 His, which can bind in the cavity created by H64A and participate in catalysis (see Carter and Wells, 1987, “Engineering enzyme specificity by ‘substrate-assisted catalysis,’” Science 237:394-399). By this model in ACE2, a bulky H345L mutation would be far more disruptive than H345A.

### Example 8. Functional Characterization of Enzymatically Active ACE2 Fusion Protein Linker Variants

The strengths of spike RBD binding and virus neutralization for enzymatically active versions of the lead ACE2 receptor trap, CVD313 (K31F/N33D/H34S/E35Q/H345L), having various linkers between the ACE2(740) domain and the dimerization domain were evaluated. The ACE2 receptor trap variants are described in Table 9. The CVD433 variant includes an extra disulfide bond in the linker sequence for stability.

TABLE 9
Sequences used in receptor trap variants with enzymatically active ACE2.

| Receptor | ACE2(740) domain | | |
|---|---|---|---|
| trap | mutations (positions | | Dimerization |
| variant | relative to SEQ ID NO: 1) | Linker | domain |
| CVD430 | K31F/N33D/H34S/E35Q | GSSGGGGSGGGGSGGGGSGGGG | Human IgG1 hinge |
| (SEQ ID | (SEQ ID NO: 38) | (SEQ ID NO: 10) | and Fc domain |
| NO: 77) | | | (SEQ ID NO: 7) |
| CVD431 | K31F/N33D/H34S/E35Q | GSSGGGGSGGGGSGGGG | Human IgG1 hinge |
| (SEQ ID | (SEQ ID NO: 38) | (SEQ ID NO: 11) | and Fc domain |
| NO: 78) | | | (SEQ ID NO: 7) |
| CVD432 | K31F/N33D/H34S/E35Q | GSSGGGGSGGGG | Human IgG1 hinge |
| (SEQ ID | (SEQ ID NO: 38) | (SEQ ID NO: 12) | and Fc domain |
| NO: 79) | | | (SEQ ID NO: 7) |
| CVD433 | K31F/N33D/H34S/E35Q | CSGGGGSGGGG | Human IgG1 hinge |
| (SEQ ID | (SEQ ID NO: 38) | (SEQ ID NO: 13) | and Fc domain |
| NO: 80) | | | (SEQ ID NO: 7) |
| CVD435 | K31F/N33D/H34S/E35Q | GS | Truncated human |
| (SEQ ID | Truncated at amino acid | | IgG1 hinge and Fc |
| NO: 81) | 729 relative to SEQ ID | | domain (SEQ ID |
| | NO: 1 | | NO: 40) |
| | (SEQ ID NO: 39) | | |

The enzymatically active ACE2 receptor trap linker variants, along with CVD293, described above, were tested for viral neutralization ability on pseudotyped SARS-CoV-2 according to the methods described in Example 1. Each of the linker variants (CVD430, CVD431, CVD432, CVD433, and CVD435) were able to effectively neutralize pseudotyped SARS-CoV-2 (data not shown), with similar potency to the enzymatically inactive variants (e.g., CVD293, CVD313, etc.; see Table 7 and Example 6).

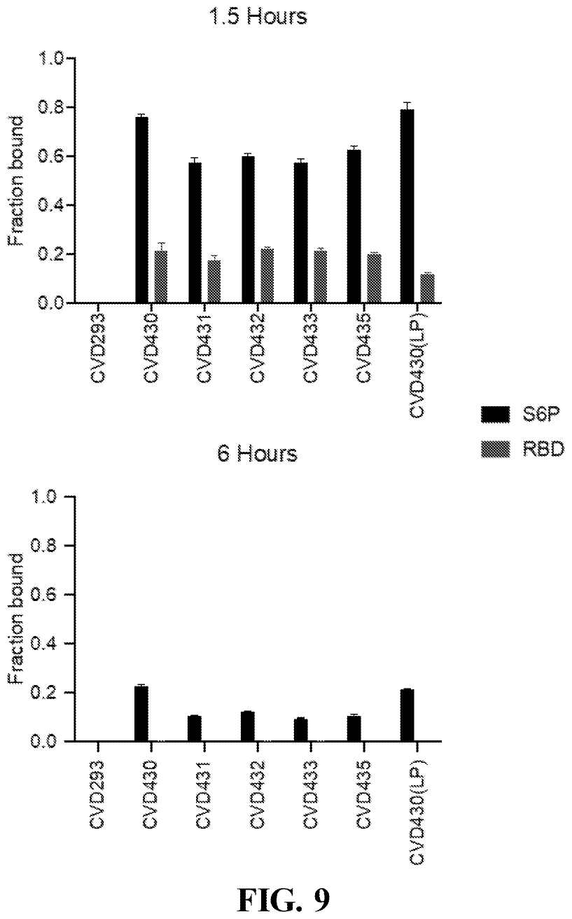

The ACE2 receptor trap variants, along with CVD293 and CVD430(LP) were also tested for relative binding strength to spike protein (S6P) and RBD, using the off rate measurement technique described in Example 1. CVD430(LP) has the same sequence as CVD430, but it was produced in transient CHO cells by LakePharma, rather than in Expi293 cells as described in Example 1. All variants, aside from CVD293, showed relatively similar binding to S6P (half life around 1.5-2 hr) and RBD (half life around 0.5-0.75 hr) (FIG. 9).

### Example 9. Binding of Enzymatically Active ACE2 Fusion Protein to Spike Proteins from SARS-CoV-2 Variants

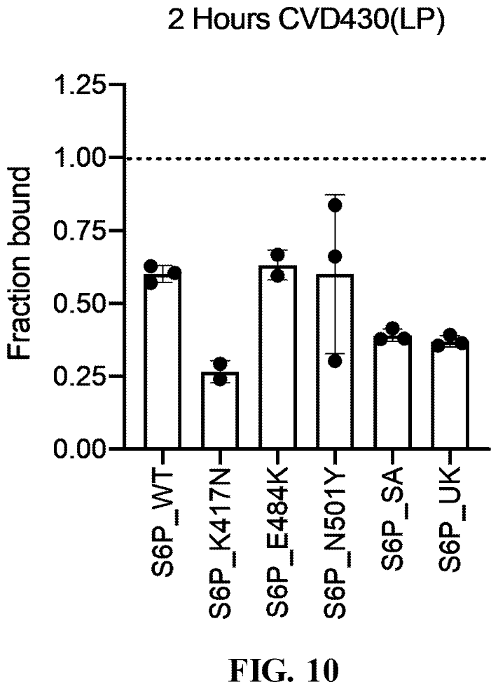

The ACE2 receptor trap construct CVD430, described in Example 8, was also tested for binding to various mutated spike proteins, including those from the UK and South Africa SARS-CoV-2 variants, using the off rate measurement technique described in Example 1. The receptor trap protein showed relatively similar binding (half life around 1-3 hr) for all spike protein variants tested (FIG. 10).

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

The following copending commonly owned patent applications are incorporated by reference in their entirety for all purposes:
DETECTION ASSAY FOR ANTI-SARS-COV-2 ANTIBODIES, Application No. PCT/US, filed May 11, 2021 (attorney docket number 103182-1244658-005510WO), and
DETECTION ASSAY FOR SARS-COV-2 VIRUS, Application No. PCT/US, filed May 11, 2021 (attorney docket number 103182-1244642-004910WO).

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

SEQUENCES

[SEQ ID NO: 1]-Full-length ACE2 (aa 1-805)
MSSSSWLLLSLVAVTAAQSTIEEQAKTFLDKFNHEAEDLFYQSSLASWNYNTNITEE
NVQNMNNAGDKWSAFLKEQSTLAQMYPLQEIQNLTVKLQLQALQQNGSSVLSEDK
SKRLNTILNTMSTIYSTGKVCNPDNPQECLLLEPGLNEIMANSLDYNERLWAWESWR
SEVGKQLRPLYEEYVVLKNEMARANHYEDYGDYWRGDYEVNGVDGYDYSRGQLI
EDVEHTFEEIKPLYEHLHAYVRAKLMNAYPSYISPIGCLPAHLLGDMWGRFWTNLYS
LTVPFGQKPNIDVTDAMVDQAWDAQRIFKEAEKFFVSVGLPNMTQGFWENSMLTDP
GNVQKAVCHPTAWDLGKGDFRILMCTKVTMDDFLTAHHEMGHIQYDMAYAAQPF
LLRNGANEGFHEAVGEIMSLSAATPKHLKSIGLLSPDFQEDNETEINFLLKQALTIVGT
LPFTYMLEKWRWMVFKGEIPKDQWMKKWWEMKREIVGVVEPVPHDETYCDPASL
FHVSNDYSFIRYYTRTLYQFQFQEALCQAAKHEGPLHKCDISNSTEAGQKLFNMLRL
GKSEPWTLALENVVGAKNMNVRPLLNYFEPLFTWLKDQNKNSFVGWSTDWSPYAD
QSIKVRISLKSALGDKAYEWNDNEMYLFRSSVAYAMRQYFLKVKNQMILFGEEDVR
VANLKPRISFNFFVTAPKNVSDIIPRTEVEKAIRMSRSRINDAFRLNDNSLEFLGIQPTL
GPPNQPPVSIWLIVFGVVMGVIVVGIVILIFTGIRDRKKKNKARSGENPYASIDISKGE
NNPGFQNTDDVQTSF
[SEQ ID NO: 7]-human IgG1 hinge and Fc domain
EPKSCDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSL
SLSPGK
[SEQ ID NO: 26]-Wild-type spike protein
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSI
IAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPS
KPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAHFPREGVF
VSNGTHWFVTQRNFYEPQUITTDNTFVSGNCDVVIGIVNNTVYDPLQPELDSFKEELD
KYFKNHTSPDVDLGDISGINASVVNIQKEIDRLNEVAKNLNESLIDLQELGKYEQYIK
WPWYIWLGFIAGLIAIVMVTIMLCCMTSCCSCLKGCCSCGSCCKFDEDDSEPVLKGV
KLHYT
[SEQ ID NO: 28]-Modified spike protein
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTDA
VRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPASVASQSIIAY
TMSLGAENSVAYSNNSIAIPTNFTISVTTEILPVSMTKTSVDCTMYICGDSTECSNLLL
QYGSFCTQLNRALTGIAVEQDKNTQEVFAQVKQIYKTPPIKDFGGFNFSQILPDPSKPS
KRSFIEDLLFNKVTLADAGFIKQYGDCLGDIAARDLICAQKFNGLTVLPPLLTDEMIA
QYTSALLAGTITSGWTFGAGAALQIPFAMQMAYRFNGIGVTQNVLYENQKLIANQF
NSAIGKIQDSLSSTASALGKLQDVVNQNAQALNTLVKQLSSNFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQUITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWPSG
RLVPRGSPGSGYIPEAPRDGQAYVRKDGEWVLLSTFLGHHHHHH

1. A recombinant ACE2 polypeptide comprising a soluble ACE2 receptor ectodomain polypeptide comprising an amino acid sequence having at least 90% sequence identity to the amino acid sequence as set forth in SEQ ID NO: 2 or 3 and comprising at least one amino acid residue substitutions selected from the group consisting of Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, and Q101R, wherein the residues are numbered with reference to SEQ ID NO:1.

2. The recombinant ACE2 polypeptide of claim 1, wherein the soluble ACE2 receptor ectodomain polypeptide comprises at least two amino acid residue substitutions selected from the group consisting of Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, and Q101R, wherein the residues are numbered with reference to SEQ ID NO:1.

3. The recombinant ACE2 polypeptide of claim 1, wherein the soluble ACE2 receptor ectodomain polypeptide comprises amino acid residue substitutions:
i. K31F, N33D, H34S, and E35Q;
ii. K31F, N33D, H34A, E35Q, N49D, N51S, N53S, E57G, and N64D;
iii. T27A, K31F, N33D, H34S, E35Q, N61D, K68R, and L79P;
iv. S19P, N33S, H34V, F40L, N49D, and L100P;
v. K31F, N33D, H34S, E35Q, W69R, and Q76R;
vi. Q18R, K31F, N33D, H34S, E35Q, W69R, and Q76R;
vii. Q18R, K31F, N33D, H34S, E35Q, W69V, and Q76R;
viii. Q18R, K31F, N33D, H34S, E35Q, W69K, and Q76R;
ix. Q18R, K31F, N33D, H34S, E35Q, W69I, and Q76R;
x. T27A, H34A, N49S, V59A, N63S, K68R, E75G, N90Q, and Q103R;
xi. K31F, N33D, H34T, N53D, W69R, and E75K;
xii. S19P, K26R, T27A, H34A, S44G, and M62T;
xiii. K31F, H34I, E35Q, and N90Q;
xiv. A25V, T27A, H34A, and F40D;
xv. K31Y, W69V, L79T, and L91P;
xvi. T27Y, H34A, and N90Q;
xvii. S19P, Q42L, L79T, and N90Q;
xviii. K31F, H34I, E35Q; or
xix. H34V and N90Q,
wherein the residues are numbered with reference to SEQ ID NO:1.

4. The recombinant ACE2 polypeptide of claim 1, wherein the soluble ACE2 receptor ectodomain polypeptide comprises amino acid residue substitutions H374N and H378N.

5. The recombinant ACE2 polypeptide of claim 1, wherein the soluble ACE2 receptor ectodomain polypeptide comprises amino acid residue substitution H345L.

6. A fusion protein comprising the recombinant ACE2 polypeptide of claim 1 fused to a dimerization domain.

7. The fusion protein of claim 6, wherein the recombinant ACE2 polypeptide is fused to the dimerization domain via a peptide linker.

8. The fusion protein of claim 6, wherein the dimerization domain comprises an Fc domain.

9. A recombinant nucleic acid encoding the recombinant ACE2 polypeptide protein of claim 1 or the fusion protein of claim 6.

10. A DNA construct comprising a promoter operably linked to the recombinant nucleic acid of claim 9.

11. The DNA construct of claim 10, wherein the promoter is a heterologous promoter.

12. A vector comprising the DNA construct of claim 10.

13. A host cell comprising the recombinant nucleic acid of claim 9.

14. A host cell comprising the DNA construct of claim 10.

15. A host cell comprising the vector of claim 12.

16. The host cell of claim 13, wherein the host cell is a eukaryotic cell.

17. A composition comprising a dimer of the recombinant ACE2 polypeptide of claim 1.

18. A method of producing a recombinant ACE2 polypeptide comprising culturing the host cell of claim 13 under conditions sufficient for the production of the recombinant ACE2 polypeptide by the host cell.

19. A pharmaceutical preparation comprising:
(a) the recombinant ACE2 polypeptide of claim 1 or the fusion protein of claim 6; and
(b) a pharmaceutically acceptable carrier.

20. A method for treating a subject infected with a SARS-CoV-2 virus or having symptoms suggestive of a SARS-CoV-2 infection, the method comprising administering to a subject a therapeutically effective amount of the pharmaceutical preparation of claim 19.

21. The method of claim 20, wherein the subject has a confirmed SARS-CoV-2 infection.

22. A method for treating a subject exposed to a SARS-CoV-2 virus or at risk of exposure to SARS-CoV-2 virus, the method comprising administering to a subject a therapeutically effective amount of the pharmaceutical preparation of claim 19.

23. The method of claim 20, wherein the subject is human.

24. The method of claim 20, wherein the pharmaceutical preparation is administered intravenously.

25. The method of claim 20, wherein the pharmaceutical preparation is administered at least once per day.

26. (canceled)

27. The recombinant ACE2 polypeptide, fusion protein, and/or composition of claim 26, wherein the recombinant ACE2 polypeptide, the fusion protein, and/or the composition has greater than 180-fold higher affinity for SARS-CoV-2 spike RBD as compared to wild-type human ACE2 protein ectodomain.

28-29. (canceled)

30. A composition comprising the fusion protein of claim 1.

31. The recombinant ACE2 polypeptide of claim 1, wherein the recombinant ACE2 polypeptide, has increased binding affinity for SARS-CoV-2 spike RBD and/or has increased efficacy in neutralizing SARS-CoV-2 virus relative to wild-type human ACE2 protein ectodomain.

32. The fusion protein of claim 6, wherein the fusion protein has increased binding affinity for SARS-CoV-2 spike RBD and/or has increased efficacy in neutralizing SARS-CoV-2 virus relative to wild-type human ACE2 protein ectodomain.

33. The composition of claim 17, wherein the fusion protein has increased binding affinity for SARS-CoV-2 spike RBD and/or has increased efficacy in neutralizing SARS-CoV-2 virus relative to wild-type human ACE2 protein ectodomain.

34. The recombinant ACE2 polypeptide of claim 31, wherein the recombinant ACE2 polypeptide has greater than 25-fold higher neutralization efficacy for SARS-CoV-2 virus compared to wild-type human ACE2 protein ectodomain.

35. The fusion protein of claim 32, wherein the fusion has greater than 25-fold higher neutralization efficacy for SARS-CoV-2 virus compared to wild-type human ACE2 protein ectodomain.

36. The composition of claim 33, wherein the fusion has greater than 25-fold higher neutralization efficacy for SARS-CoV-2 virus compared to wild-type human ACE2 protein ectodomain.
